SEQUENCE LISTING (96 sequences)

SEQ ID NO: 1 - protein, 16 aa, Artificial Sequence
  Description of Artificial Sequence Synthetic peptide
RSSQSLVHSNGNTYLH

SEQ ID NO: 2 - protein, 16 aa, Artificial Sequence
  Description of Artificial Sequence Synthetic peptide
RSSQSLENSNGDTYLN

SEQ ID NO: 3 - protein, 16 aa, Artificial Sequence
  Description of Artificial Sequence Synthetic peptide
RSSQSLVHSNGNTYLH

SEQ ID NO: 4 - protein, 16 aa, Artificial Sequence
  Description of Artificial Sequence Synthetic peptide
RSSQNIVQSNGNTYLE

SEQ ID NO: 5 - protein, 10 aa, Artificial Sequence
  Description of Artificial Sequence Synthetic peptide
RASSSIFYVD

SEQ ID NO: 6 - protein, 10 aa, Artificial Sequence
  Description of Artificial Sequence Synthetic peptide
SASSSIFYVD

SEQ ID NO: 7 - protein, 7 aa, Artificial Sequence
  Description of Artificial Sequence Synthetic peptide
KVSNRLS

SEQ ID NO: 8 - protein, 7 aa, Artificial Sequence
  Description of Artificial Sequence Synthetic peptide
RVSNRFS

SEQ ID NO: 9 - protein, 7 aa, Artificial Sequence
  Description of Artificial Sequence Synthetic peptide
KVFHRFS

SEQ ID NO: 10 - protein, 7 aa, Artificial Sequence
  Description of Artificial Sequence Synthetic peptide
DTSKLAS

SEQ ID NO: 11 - protein, 9 aa, Artificial Sequence
  Description of Artificial Sequence Synthetic peptide
SQTTHVPYT

SEQ ID NO: 12 - protein, 9 aa, Artificial Sequence
  Description of Artificial Sequence Synthetic peptide
LQVSHVPWT

SEQ ID NO: 13 - protein, 9 aa, Artificial Sequence
  Description of Artificial Sequence Synthetic peptide
SQSTHVPRT

SEQ ID NO: 14 - protein, 8 aa, Artificial Sequence
  Description of Artificial Sequence Synthetic peptide
SQSTHVLT

SEQ ID NO: 15 - protein, 9 aa, Artificial Sequence
  Description of Artificial Sequence Synthetic peptide
FQGSHVPWT

SEQ ID NO: 16 - protein, 9 aa, Artificial Sequence
  Description of Artificial Sequence Synthetic peptide
FQGSYVPWT

SEQ ID NO: 17 - protein, 9 aa, Artificial Sequence
  Description of Artificial Sequence Synthetic peptide
QQWSSNPPT

SEQ ID NO: 18 - protein, 10 aa, Artificial Sequence
  Description of Artificial Sequence Synthetic peptide
GYTFTNYGMN

SEQ ID NO: 19 - protein, 10 aa, Artificial Sequence
  Description of Artificial Sequence Synthetic peptide
GYTFTNYWIH

SEQ ID NO: 20 - protein, 10 aa, Artificial Sequence
  Description of Artificial Sequence Synthetic peptide
GYTFTNYFLY

SEQ ID NO: 21 - protein, 10 aa, Artificial Sequence
  Description of Artificial Sequence Synthetic peptide
DYTFTNYYIH

SEQ ID NO: 22 - protein, 10 aa, Artificial Sequence
  Description of Artificial Sequence Synthetic peptide
GYTFTNYWMH

SEQ ID NO: 23 - protein, 10 aa, Artificial Sequence
  Description of Artificial Sequence Synthetic peptide
GYSFTGYYMH

SEQ ID NO: 24 - protein, 17 aa, Artificial Sequence
  Description of Artificial Sequence Synthetic peptide
WININTGEPTYAEDFKG

SEQ ID NO: 25 - protein, 17 aa, Artificial Sequence
  Description of Artificial Sequence Synthetic peptide
YIDPNTVYTDYNQRFED

SEQ ID NO: 26 - protein, 17 aa, Artificial Sequence
  Description of Artificial Sequence Synthetic peptide
DINPNAGSTNLNERFKS

SEQ ID NO: 27 - protein, 17 aa, Artificial Sequence
  Description of Artificial Sequence Synthetic peptide
WIYPGNNNNKYNEKFKG

SEQ ID NO: 28 - protein, 17 aa, Artificial Sequence
  Description of Artificial Sequence Synthetic peptide
YIDPRTAYTEYNQKFKD

SEQ ID NO: 29 - protein, 17 aa, Artificial Sequence
  Description of Artificial Sequence Synthetic peptide
YIDPRTDYSEYNQKFKD

SEQ ID NO: 30 - protein, 17 aa, Artificial Sequence
  Description of Artificial Sequence Synthetic peptide
RANPYNGGTSYNQKFKG

SEQ ID NO: 31 - protein, 9 aa, Artificial Sequence
  Description of Artificial Sequence Synthetic peptide
WARGGNFDL

SEQ ID NO: 32 - protein, 8 aa, Artificial Sequence
  Description of Artificial Sequence Synthetic peptide
GGKRGVDS

SEQ ID NO: 33 - protein, 7 aa, Artificial Sequence
  Description of Artificial Sequence Synthetic peptide
GGYTMDY

SEQ ID NO: 34 - protein, 7 aa, Artificial Sequence
  Description of Artificial Sequence Synthetic peptide
GGYTMDY

SEQ ID NO: 35 - protein, 8 aa, Artificial Sequence
  Description of Artificial Sequence Synthetic peptide
GGRVGLGY

SEQ ID NO: 36 - protein, 10 aa, Artificial Sequence
  Description of Artificial Sequence Synthetic peptide
NYGGSDAMDY

SEQ ID NO: 37 - protein, 10 aa, Artificial Sequence
  Description of Artificial Sequence Synthetic peptide
NYGSSDAMDY

SEQ ID NO: 38 - protein, 112 aa, Artificial Sequence
  Description of Artificial Sequence Synthetic peptide
DVVLTQTPLSLPVGLGDQASISCRSSQSLVHSNGNTYLHWYLQKPGQSPKLLIYKVSNRLSGVPDRFSGSGSGTDFTLRISRVEAEDLGVYFCSQTTHVPYTFGGGTELEIK

SEQ ID NO: 39 - protein, 112 aa, Artificial Sequence
  Description of Artificial Sequence Synthetic peptide
DVVMTQTPLSLPVSLGDQASISCRSSQSLENSNGDTYLNWYLQKPGQSPQLLIYRVSNRFSGVLDRFSGSGSGTDFTLQISRVEAEDLGVYFCLQVSHVPWTFGGGTNLEIK

SEQ ID NO: 40 - protein, 112 aa, Artificial Sequence; identical to SEQ ID NO: 39
  Description of Artificial Sequence Synthetic peptide

SEQ ID NO: 41 - protein, 112 aa, Artificial Sequence
  Description of Artificial Sequence Synthetic peptide
DVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPRTFGGGTKLEIK

SEQ ID NO: 42 - protein, 105 aa, Artificial Sequence
  Description of Artificial Sequence Synthetic peptide
DVLMTQTPLSLPVSLGDQASISCRSSQSLVHSNGNTYLHWYLQKPGQSPNLLIYKVSNRFSGVPDRFSGSGSGTDFTLKINRVETEDLGIYFCSQSTHVLTFGAG

SEQ ID NO: 43 - protein, 112 aa, Artificial Sequence
  Description of Artificial Sequence Synthetic peptide
DVLMTQTPLSLPVSLGDQASISCRSSQNIVQSNGNTYLEWYLQKPGQSPKLLIYKVFHRFSGVPDRFSGSGSGTDFTLKISGVEAEDLGVYYCFQGSHVPWTFGGGTRLEIK

SEQ ID NO: 44 - protein, 112 aa, Artificial Sequence
  Description of Artificial Sequence Synthetic peptide
DVLMTQTPLSLPISLGDQASISCRSSQNIVQSNGNTYLEWYLQKPGQSPKLLIYKVFHRFSGVPDRFSGSGSGTDFTLKISRVEAEDLGVYYCFQGSYVPWTFGGGTRLEIK

SEQ ID NO: 45 - protein, 106 aa, Artificial Sequence
  Description of Artificial Sequence Synthetic peptide
QIVLTQSPAIMSASPGERVTMTCRASSSIFYVDWYQQKSGTSPKRWIYDTSKLASGVPARFSGSGSGTSYSLTISSMEAEDAATYHCQQWSSNPPTFGAGTKLELK

SEQ ID NO: 46 - protein, 106 aa, Artificial Sequence
  Description of Artificial Sequence Synthetic peptide
QIVLTQSPAIMSASPGERVTMTCSASSSIFYVDWYQQKSGTSPKRWIYDTSKLASGVPARFSGSGSGTSYSLTISSMEAEDAATYHCQQWSSNPPTFGAGTKLELK

SEQ ID NO: 47 - protein, 118 aa, Artificial Sequence
  Description of Artificial Sequence Synthetic peptide
QIQLVQSGPELKKPGETAKISCKASGYTFTNYGMNWVKQAPGKDLKWMGWININTGEPTYAEDFKGRFVFSLETSAGTAYLQISNLKNEDTATYFCARWARGGNFDLWGQGTTLTVSS

SEQ ID NO: 48 - protein, 117 aa, Artificial Sequence
  Description of Artificial Sequence Synthetic peptide
QVQLQQSGAELAKPGASVKMSCKASGYTFTNYWIHWIKQRPGQGLEWIGYIDPNTVYTDYNQRFEDKATLTADKSSNTAYMQLNSLTSEDSAVYYCSRGGKRGVDSWGQGTSVTVSS

SEQ ID NO: 49 - protein, 116 aa, Artificial Sequence
  Description of Artificial Sequence Synthetic peptide
EVQLQQSGAQLVKPGTSMKLSCKASGYTFTNYFLYWVKQRPGQGLEWIGDINPNAGSTNLNERFKSKATLTVDKSSSTAYLQLSGLTSEDSAVYYCTRGGYTMDYWGQGTSVTVSS

SEQ ID NO: 50 - protein, 116 aa, Artificial Sequence
  Description of Artificial Sequence Synthetic peptide
QVQLLQSGAQLVKPGTSMKLSCKASGYTFTNYFLYWVKQRPGQGLEWIGDINPNAGSTNLNERFKSKATLTVDKSSSTAYLQLSGLTSEDSAVYYCTRGGYTMDYWGQGTSVTVSS

SEQ ID NO: 51 - protein, 109 aa, Artificial Sequence
  Description of Artificial Sequence Synthetic peptide
EVQLQQSGPEVVKPGASVRISCKASDYTFTNYYIHWVRQRPGQGLEWIGWIYPGNNNNKYNEKFKGKATLTEDTSSSTAYMQLSSLTSEDSAVYFCARGGYTMDYWGQG

SEQ ID NO: 52 - protein, 117 aa, Artificial Sequence
  Description of Artificial Sequence Synthetic peptide
QVQLQQSGAELAKPGASVQMSCKASGYTFTNYWMHWVKQRSGQGLEWIGYIDPRTAYTEYNQKFKDKATLTADKSSSTAYMRLSSLTSEDSAVYYCVGGGRVGLGYWGHGSSVTVSS

SEQ ID NO: 53 - protein, 117 aa, Artificial Sequence
  Description of Artificial Sequence Synthetic peptide
EVQLQQSGAELAKPGASVKMSCKASGYTFTNYWMHWVKQRPGQGLEWIGYIDPRTDYSEYNQKFKDKATLTADKSSSTAYMQLSSLTSEDSAVYFCAGGGRVGLGYWGHGSSVTVSS

SEQ ID NO: 54 - protein, 119 aa, Artificial Sequence
  Description of Artificial Sequence Synthetic peptide
EVQLQQSGPDLVKPGASVKISCKASGYSFTGYYMHWVKQSHGKSLEWIGRANPYNGGTSYNQKFKGKAILTVDKSSSTAYMELRSLTSEDSAVYYCARNYGGSDAMDYWGQGTSITVAS

SEQ ID NO: 55 - protein, 119 aa, Artificial Sequence
  Description of Artificial Sequence Synthetic peptide
EVQLQQSGPDLVKPGASVKISCKASGYSFTGYYMHWVKQSHGKSLEWIGRANPYNGGTSYNQKFKGKAILTVDKSSSTAYMELRSLTSEDSAVYYCARNYGSSDAMDYWGQGTSITVAS

SEQ ID NO: 56 - protein, 219 aa, Artificial Sequence
  Description of Artificial Sequence Synthetic peptide
DVVMTQTPLSLPVSLGDQASISCRSSQSLENSNGDTYLNWYLQKPGQSPQLLIYRVSNRFSGVLDRFSGSGSGTDFTLQISRVEAEDLGVYFCLQVSHVPWTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 447 aa, Artificial Sequence
  Description of Artificial Sequence Synthetic peptide
QVQLQQSGAELAKPGASVKMSCKASGYTFTNYWIHWIKQRPGQGLEWIGYIDPNTVYTDYNQRFEDKATLTADKSSNTAYMQLNSLTSEDSAVYYCSRGGKRGVDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 443 aa, Artificial Sequence
  Description of Artificial Sequence Synthetic peptide
QVQLQQSGAELAKPGASVKMSCKASGYTFTNYWIHWIKQRPGQGLEWIGYIDPNTVYTDYNQRFEDKATLTADKSSNTAYMQLNSLTSEDSAVYYCSRGGKRGVDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 219 aa, Artificial Sequence
  Description of Artificial Sequence Synthetic peptide
DVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 446 aa, Artificial Sequence
  Description of Artificial Sequence Synthetic peptide
QVQLLQSGAQLVKPGTSMKLSCKASGYTFTNYFLYWVKQRPGQGLEWIGDINPNAGSTNLNERFKSKATLTVDKSSSTAYLQLSGLTSEDSAVYYCTRGGYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 442 aa, Artificial Sequence
  Description of Artificial Sequence Synthetic peptide
QVQLLQSGAQLVKPGTSMKLSCKASGYTFTNYFLYWVKQRPGQGLEWIGDINPNAGSTNLNERFKSKATLTVDKSSSTAYLQLSGLTSEDSAVYYCTRGGYTMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 219 aa, Artificial Sequence
  Description of Artificial Sequence Synthetic peptide
DVVLTQTPLSLPVGLGDQASISCRSSQSLVHSNGNTYLHWYLQKPGQSPKLLIYKVSNRLSGVPDRFSGSGSGTDFTLRISRVEAEDLGVYFCSQTTHVPYTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 448 aa, Artificial Sequence
  Description of Artificial Sequence Synthetic peptide
QIQLVQSGPELKKPGETAKISCKASGYTFTNYGMNWVKQAPGKDLKWMGWININTGEPTYAEDFKGRFVFSLETSAGTAYLQISNLKNEDTATYFCARWARGGNFDLWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 444 aa, Artificial Sequence
  Description of Artificial Sequence Synthetic peptide
QIQLVQSGPELKKPGETAKISCKASGYTFTNYGMNWVKQAPGKDLKWMGWININTGEPTYAEDFKGRFVFSLETSAGTAYLQISNLKNEDTATYFCARWARGGNFDLWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 112 aa, Artificial Sequence
  Description of Artificial Sequence Synthetic peptide
DIVMTQSPLSLPVTPGEPASISCRSSQSLENSNGDTYLNWYLQKPGQSPRLLIYRVSNRFSGVPDRFSGSGSGTDFTLKISRVEADDVGIYYCLQVSHVPWTFGQGTKLEIK

SEQ ID NO: 66 - protein, 112 aa, Artificial Sequence
  Description of Artificial Sequence Synthetic peptide
DIVMTQSPLSLPVTPGEPASISCRSSQSLVHSNGNTYLHWYLQKPGQSPRLLIYKVSNRLSGVPDRFSGSGSGTDFTLKISRVEADDVGIYYCSQTTHVPYTFGQGTKLEIK

SEQ ID NO: 67 - protein, 112 aa, Artificial Sequence
  Description of Artificial Sequence Synthetic peptide
DVVMTQSPLSLPVTLGQPASISCRSSQSLVHSNGNTYLHWFQQRPGQSPRRLIYKVSNRLSGVPDRFSGSGSGTDFTLKISRVEAEDVGVYYCSQTTHVPYTFGQGTKLEIK

SEQ ID NO: 68 - protein, 112 aa, Artificial Sequence
  Description of Artificial Sequence Synthetic peptide
DIVMTQSPDSLAVSLGERATINCRSSQSLVHSNGNTYLHWYQQKPGQPPKLLIYKVSNRLSGVPDRFSGSGSGTDFTLTISSLQAEDVAVYYCSQTTHVPYTFGQGTKLEIK

SEQ ID NO: 69 - protein, 117 aa, Artificial Sequence
  Description of Artificial Sequence Synthetic peptide
QVQLVQSGAEVKKPGASVQVSCKASGYTFTNYWIHWLRQAPGQGLEWMGYIDPNTVYTDYNQRFEDRVTMTSDTSISTAYMELSSLRSDDTAVYYCARGGKRGVDSWGQATLVTVSS

SEQ ID NO: 70 - protein, 118 aa, Artificial Sequence
  Description of Artificial Sequence Synthetic peptide
QVQLVQSGAEVKKPGASVQVSCKASGYTFTNYGMNWLRQAPGQGLEWMGWININTGEPTYAEDFKGRVTMTSDTSISTAYMELSSLRSDDTAVYYCARWARGGNFDLWGQATLVTVSS

SEQ ID NO: 71 - protein, 118 aa, Artificial Sequence
  Description of Artificial Sequence Synthetic peptide
EVQLVQSGAEVKKPGATVKISCKVSGYTFTNYGMNWVQQAPGKGLEWMGWININTGEPTYAEDFKGRVTITADTSTDTAYMELSSLRSEDTAVYYCATWARGGNFDLWGQGTTVTVSS

SEQ ID NO: 72 - protein, 118 aa, Artificial Sequence
  Description of Artificial Sequence Synthetic peptide
EVQLVQSGAEVKKPGESLKISCKGSGYTFTNYGMNWVRQMPGKGLEWMGWININTGEPTYAEDFKGQVTISADKSISTAYLQWSSLKASDTAMYYCARWARGGNFDLWGQGTTVTVSS

SEQ ID NO: 73 - protein, 118 aa, Artificial Sequence
  Description of Artificial Sequence Synthetic peptide
QVQLVQSGAEVKKPGASVKVSCKASGYTFTNYGMNWVRQAPGQGLEWMGWININTGEPTYAEDFKGRVTMTTDTSTSTAYMELRSLRSDDTAVYYCARWARGGNFDLWGQGTTVTVSS

SEQ ID NO: 74 - protein, 219 aa, Artificial Sequence
  Description of Artificial Sequence Synthetic peptide
DIVMTQSPLSLPVTPGEPASISCRSSQSLENSNGDTYLNWYLQKPGQSPRLLIYRVSNRFSGVPDRFSGSGSGTDFTLKISRVEADDVGIYYCLQVSHVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 219 aa, Artificial Sequence
  Description of Artificial Sequence Synthetic peptide
DIVMTQSPLSLPVTPGEPASISCRSSQSLVHSNGNTYLHWYLQKPGQSPRLLIYKVSNRLSGVPDRFSGSGSGTDFTLKISRVEADDVGIYYCSQTTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 219 aa, Artificial Sequence
  Description of Artificial Sequence Synthetic peptide
DVVMTQSPLSLPVTLGQPASISCRSSQSLVHSNGNTYLHWFQQRPGQSPRRLIYKVSNRLSGVPDRFSGSGSGTDFTLKISRVEAEDVGVYYCSQTTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 219 aa, Artificial Sequence
  Description of Artificial Sequence Synthetic peptide
DIVMTQSPDSLAVSLGERATINCRSSQSLVHSNGNTYLHWYQQKPGQPPKLLIYKVSNRLSGVPDRFSGSGSGTDFTLTISSLQAEDVAVYYCSQTTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 443 aa, Artificial Sequence
  Description of Artificial Sequence Synthetic peptide
QVQLVQSGAEVKKPGASVQVSCKASGYTFTNYWIHWLRQAPGQGLEWMGYIDPNTVYTDYNQRFEDRVTMTSDTSISTAYMELSSLRSDDTAVYYCARGGKRGVDSWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 79 - protein, 770 aa, Artificial Sequence
  Description of Artificial Sequence Synthetic peptide
QVQLVQSGAEVKKPGASVQVSCKASGYTFTNYGMNWLRQAPGQGLEWMGWININTGEPTYAEDFKGRVTMTSDTSISTAYMELSSLRSDDTAVYYCARWARGGNFDLWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 444 aa, Artificial Sequence
  Description of Artificial Sequence Synthetic peptide
EVQLVQSGAEVKKPGATVKISCKVSGYTFTNYGMNWVQQAPGKGLEWMGWININTGEPTYAEDFKGRVTITADTSTDTAYMELSSLRSEDTAVYYCATWARGGNFDLWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 81 - protein, 444 aa, Artificial Sequence
  Description of Artificial Sequence Synthetic peptide
EVQLVQSGAEVKKPGESLKISCKGSGYTFTNYGMNWVRQMPGKGLEWMGWININTGEPTYAEDFKGQVTISADKSISTAYLQWSSLKASDTAMYYCARWARGGNFDLWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 444 aa, Artificial Sequence
  Description of Artificial Sequence Synthetic peptide
QVQLVQSGAEVKKPGASVKVSCKASGYTFTNYGMNWVRQAPGQGLEWMGWININTGEPTYAEDFKGRVTMTTDTSTSTAYMELRSLRSDDTAVYYCARWARGGNFDLWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 83 - protein, 330 aa, Artificial Sequence [sequence omitted]

SEQ ID NO: 84 - protein, 330 aa, Artificial Sequence
  Description of Artificial Sequence Synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 326 aa, Artificial Sequence
  Description of Artificial Sequence Synthetic peptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 377 aa, Artificial Sequence
  Description of Artificial Sequence Synthetic peptide
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 87 - protein, 326 aa, Artificial Sequence
  Description of Artificial Sequence Synthetic peptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 88 - protein, 326 aa, Artificial Sequence
  Description of Artificial Sequence Synthetic peptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 89 - protein, 327 aa, Artificial Sequence
  Description of Artificial Sequence Synthetic peptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 326 aa, Artificial Sequence
  Description of Artificial Sequence Synthetic peptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 91 - protein, 107 aa, Artificial Sequence [sequence omitted]

SEQ ID NO: 92 - protein, 329 aa, Artificial Sequence
  Description of Artificial Sequence Synthetic peptide
ARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 93 - protein, 107 aa, Artificial Sequence
  Description of Artificial Sequence Synthetic peptide
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 94 - protein, 323 aa, Artificial Sequence
  Description of Artificial Sequence Synthetic peptide
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 95 - protein, 104 aa, Artificial Sequence
  Description of Artificial Sequence Synthetic peptide
RDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 96 - protein, 293 aa, Artificial Sequence
  Description of Artificial Sequence Synthetic peptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

(19) United States
(12) Patent Application Publication
(10) Pub. No.: US 2021/0324075 A1
(43) Pub. Date: Oct. 21, 2021

(54) THERAPEUTIC CD47 ANTIBODIES

(71) Applicant: Arch Oncology, Inc., St. Louis, MO (US)

(72) Inventors: Robyn PURO, St. Louis, MO (US); Pamela T. MANNING, Chesterfield, MO (US); Robert W. KARR, Frontenac, MO (US); Juan C. ALMAGRO, Cambridge, MA (US)

(21) Appl. No.: 17/267,710

(22) PCT Filed: Aug. 13, 2019
(86) PCT No.: PCT/US2019/046378

§ 371 Date: Feb. 10, 2021

Related U.S. Application Data
(60) Provisional application No. 62/718,203, filed on Aug. 13, 2018.

(51) Int. Cl. C07K 16/28; A61P 35/00; G01N 33/574

(52) U.S. Cl. CPC C07K 16/2803; A61P 35/00; G01N 33/57492; C07K 2317/76; C07K 2317/73; A61K 2039/505; C07K 2317/20; C07K 2317/24; C07K 2317/732; C07K 2317/52; C07K 2317/565

(57) ABSTRACT

Provided are murine, chimeric, and humanized anti-CD47 monoclonal antibodies (anti-CD47 mAbs) with distinct functional profiles, methods to generate anti-CD47 mAbs, and methods of using these anti-CD47 mAbs as therapeutics for the prevention and treatment of solid and hematological cancers, ischemia-reperfusion injury, cardiovascular diseases, autoimmune diseases, inflammatory diseases, and as diagnostics for determining the level of CD47 in tissue samples.

### PRIORITY DATA

This application claims the benefit of U.S. Provisional Application No. 62/718,203 filed Aug. 13, 2018, the disclosure of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 13, 2019, is named VLX0009-401-PC-Sequence Listing_ST25.txt and is 135,287 bytes in size.

### FIELD OF THE DISCLOSURE

This disclosure is related generally to anti-CD47 monoclonal antibodies (anti-CD47 mAbs) with distinct functional profiles as described herein, methods to generate anti-CD47 mAbs, and to methods of using these anti-CD47 mAbs as therapeutics for the prevention and treatment of solid and hematological cancers, ischemia-reperfusion injury, cardiovascular diseases, autoimmune diseases, inflammatory diseases, or as diagnostics for determining the level of CD47 in tissue samples.

### BACKGROUND OF THE DISCLOSURE

CD47 is a cell surface receptor comprised of an extracellular IgV set domain, a 5 transmembrane domain, and a cytoplasmic tail that is alternatively spliced. Two ligands bind CD47: signal inhibitory receptor protein α (SIRPα) and thrombospondin-1 (TSP1). CD47 expression and/or activity have been implicated in a number of diseases and disorders. Accordingly, there exists a need for therapeutic compositions and methods for treating diseases and conditions associated with CD47 in humans and animals, including the prevention and treatment of solid and hematological cancers, ischemia-reperfusion injury (IRI), cardiovascular diseases, or an autoimmune or inflammatory disease. There also exists a need for diagnostic compositions and methods for determining the level of CD47 expression in tumor samples.

The present disclosure describes murine, chimeric, and humanized anti-CD47 mAbs with distinct functional profiles. These antibodies possess one or more distinct combinations of properties selected from the following: 1) exhibit cross-reactivity with one or more species homologs of CD47; 2) block the interaction between CD47 and its ligand SIRPα; 3) do not block the interaction between CD47 and its ligand SIRPα; 4) increase phagocytosis of human tumor cells, 4) induce death of susceptible human tumor cells; 5) do not induce cell death of human tumor cells; 5) reverse TSP1 inhibition of the nitric oxide (NO) pathway and/or 6) do not reverse TSP1 inhibition of the NO pathway. The antibodies of the disclosure are useful in various therapeutic methods for treating diseases and conditions associated with CD47 in humans and animals, including the prevention and treatment of solid and hematological cancers, autoimmune diseases, inflammatory diseases, IRI, and cardiovascular diseases. The antibodies of the disclosure are also useful as diagnostics to determine the level of CD47 expression in tissue samples. Embodiments of the disclosure include isolated antibodies and immunologically active binding fragments thereof; pharmaceutical compositions comprising one or more of the anti-CD47 monoclonal antibodies, preferably chimeric or humanized forms of said antibodies; methods of therapeutic use of such anti-CD47 monoclonal antibodies; and cell lines that produce these anti-CD47 monoclonal antibodies.

The embodiments of the disclosure include the mAbs, or antigen-binding fragments thereof, which are defined by reference to specific structural characteristics i.e. specified amino acid sequences of either the CDRs or entire heavy chain or light chain variable domains. All of these antibodies bind to CD47.

The monoclonal antibodies, or antigen binding fragments thereof may comprise at least one, usually at least three, CDR sequences as provided herein, usually in combination with framework sequences from a human variable region or as an isolated CDR peptide. In some embodiments, the antibody comprises at least one light chain comprising three light chain CDR sequences provided in a variable region framework, which may be, without limitation, a murine or human variable region framework, and at least one heavy chain comprising three heavy chain CDR sequences provided in a variable region framework, which may be, without limitation, a human or murine variable region framework.

In another embodiment, the monoclonal antibody, or antigen binding fragment thereof specifically also binds to non-human primate CD47, wherein non-human primate may include, but is not limited to, cynomolgus monkey, green monkey, rhesus monkey and squirrel monkey.

In yet another embodiment, the monoclonal antibody, or antigen binding fragment thereof binds to human, non-human primate, mouse, rabbit, and rat CD47.

Various forms of the anti-CD47 mAbs disclosed are contemplated herein. For example, the anti-CD47 mAbs can be full length humanized antibodies with human frameworks and constant regions of the isotypes, IgA, IgD, IgE, IgG, and IgM, more particularly, IgG1, IgG2, IgG3, IgG4, and in some cases with various mutations to alter Fc receptor function or prevent Fab arm exchange or an antibody fragment, e.g., a F(ab′)2 fragment, a F(ab) fragment, a single chain Fv fragment (scFv), etc., as disclosed herein.

The embodiments of the disclosure provide pharmaceutical or veterinary compositions comprising one or more of the anti-CD47 mAbs or fragments disclosed herein, optionally chimeric or humanized forms, and a pharmaceutically acceptable carrier, diluent, or excipient.

Prior to the present disclosure, there was a need to identify anti-CD47 mAbs that possess the functional profiles as described. The anti-CD47 mAbs of the present disclosure exhibit distinct combinations of properties, particularly combinations of properties that render the mAbs particularly advantageous or suitable for use in human therapy, particularly in the prevention and/or treatment of solid and hematological cancers, ischemia-reperfusion injury, autoimmune and/or inflammatory diseases.

Further scope of the applicability of the present disclosure will become apparent from the detailed description provided below. However, it should be understood that the detailed description and specific examples, while indicating preferred embodiments of the disclosure, are given by way of illustration only since various changes and modifications within the spirit and scope of the disclosure will become apparent to those skilled in the art from this detailed description.

### BRIEF DESCRIPTION OF THE FIGURES

The above and other aspects, features, and advantages of the present disclosure will be better understood from the following detailed descriptions taken in conjunction with the accompanying drawing(s), all of which are given by way of illustration only, and are not limitative of the present disclosure.

FIG. 1A. Binding of Murine Anti-CD47 mAbs to Murine RBCs (mRBCs). The binding of mouse anti-CD47 mAbs (Vx10 and Vx11) to mouse CD47 was determined using freshly isolated mRBCs. The mRBCs were incubated for 60 minutes at 37° C. with increasing concentrations of the mAbs, then washed and incubated for 1 hr with FITC-labeled goat anti-mouse antibody. Cells were then washed, and antibody binding was measured using flow cytometry.

FIG. 1B. Binding of Murine Anti-CD47 mAbs to Human RBCs (hRBCs). The binding of mouse anti-CD47 mAbs (Vx10 and Vx12) to human CD47 was determined using freshly isolated hRBCs. The hRBCs were incubated for 60 minutes at 37° C. with increasing concentrations of the mAbs, then washed and incubated for 1 hr with FITC-labeled goat anti-mouse antibody. Cells were then washed, and antibody binding was measured using flow cytometry.

FIG. 2A. Binding of Humanized Anti-CD47 mAbs to Human RBCs (hRBCs). The binding of humanized anti-CD47 mAbs (humVx10_01 and humVx14_07) to human CD47 was determined using freshly isolated hRBCs. The hRBCs were incubated for 60 minutes at 37° C. with increasing concentrations of the mAbs, then washed and incubated for 1 hr with FITC-labeled donkey anti-human antibody. Cells were then washed, and antibody binding was measured using flow cytometry.

FIG. 2B. Binding of Humanized Anti-CD47 mAbs to Human OV10 hCD47 Cells. The binding of humVx10_01 and humVx14_07 to human CD47 was determined using an OV10 human CD47 cell-based ELISA OV10 hCD47 cells were plated into 96 well plates and were confluent at the time of assay. Increasing concentrations of mAbs were added to the cells for 1 hr. Cells were washed and then incubated with HRP-labelled secondary antibody for 1 hr followed by addition of peroxidase substrate.

FIG. 3. Reversal of TSP1 Inhibition of NO-stimulated cGMP Production by Anti-CD47 Antibodies. Jurkat cells were incubated overnight in serum-free medium and then incubated with 10 μg/ml of the 1000 series antibodies with or without TSP1, followed by treatment with or without a NO donor. After 5 minutes, cells were lysed, and cGMP measured. The mAb Vx13 reversed the TSP1 inhibition of cGMP production by Jurkat cells whereas mAbs Vx10, Vx11, and Vx12 did not reverse TSP1 inhibition of cGMP production.

FIG. 4. Murine Anti-CD47 mAbs block SIRPα binding to CD47 on Jurkat cells. 1.5×10⁶ Jurkat cells were incubated with 5 μg/ml of Vx10, Vx11, Vx12, Vx13, or control mAb W6/32 in RPMI containing 10% media for 30 min at 37° C. An equal volume of fluorescently labeled SIRPα-Fc fusion protein was added and incubated for an additional 30 min at 37° C. Cells were washed and binding was assessed using flow cytometry. Percent binding was calculated compared to no antibody treatment.

FIG. 5A. Chimeric Anti-CD47 mAbs Increase Phagocytosis of Jurkat T Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat T cells and 1 μg/ml of the chimeric mAbs were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14⁺/CFSE+ cells in the total CD14⁺ population.

FIG. 5B. CD47 humanized mAbs Increase Phagocytosis of Jurkat T Cells by Human Macrophages. Human macrophages were plated at a concentration of 1×10⁴ cells per well in a 96 well plate and allowed to adhere for 24 hrs. 5×10⁴ CFSE (1 μM) labeled human Jurkat T cells and 1 μg/ml of the humanized mAbs were added to the macrophage cultures and incubated at 37° C. for 2 hrs. Non-phagocytosed Jurkat cells were removed and macrophage cultures were washed extensively. Macrophages were trypsinized and stained for CD14. Flow cytometry was used to determine the percentage of CD14+/CFSE+ cells in the total CD14+ population.

FIG. 6A. Induction of Cell Death in Human Jurkat T Cells by Soluble Chimeric and Humanized CD47 mAbs. Jurkat T cells (1×10⁴) were incubated with 1 μg/ml chimeric or humanized mAbs in 1 ml of RPMI media for 24 hours at 37° C. Cells were then stained with annexin V and the signal was detected by flow cytometry.

FIG. 6B. Induction of Cell Death in Human Jurkat T Cells by Soluble Chimeric and Humanized CD47 mAbs. Jurkat T cells (1×10⁴) were incubated with 1 μg/ml of a chimeric mAb or of a humanized mAb in 1 ml of RPMI media for 24 hours at 37° C. Cells were then stained with 7-AAD and the signal was detected by flow cytometry.

### DETAILED DESCRIPTION OF THE EMBODIMENTS OF THE DISCLOSURE

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

As used herein, the term “CD47”, “integrin-associated protein (IAP)”, “ovarian cancer antigen OA3”, “Rh-related antigen” and “MERG” are synonymous and may be used interchangeably.

The term “anti-CD47 antibody” refer to an antibody of the disclosure which is intended for use as a therapeutic or diagnostic agent, and therefore will typically possess the binding affinity required to be useful as a therapeutic and/or diagnostic agent.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts” with or directed against is meant that the antibody reacts with one or more antigenic determinants of the desired antigen and does not react with other polypeptides or binds at a much lower affinity (K_{d} >10⁻⁶ M). Antibodies include but are not limited to, polyclonal, monoclonal, chimeric, Fab fragments, Fab′ fragments, F(ab′)₂ fragments, single chain Fv fragments, and one-armed antibodies.

As used herein, the term “monoclonal antibody” (mAb) as applied to the present antibody compounds refers to an antibody that is derived from a single copy or clone including, for example, any eukaryotic, prokaryotic, or phage clone, and not the method by which it is produced. mAbs of the present disclosure preferably exist in a homogeneous or substantially homogeneous population. Complete mAbs contain 2 heavy chains and 2 light chains.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

As disclosed herein, “antibody compounds” refers to mAbs and antigen-binding fragments thereof. Additional antibody compounds exhibiting similar functional properties according to the present disclosure can be generated by conventional methods. For example, mice can be immunized with human CD47 or fragments thereof, the resulting antibodies can be recovered and purified, and determination of whether they possess binding and functional properties similar to or the same as the antibody compounds disclosed herein can be assessed by the methods disclosed in Examples 3-11, below. Antigen-binding fragments can also be prepared by conventional methods. Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15.

The monoclonal antibodies encompass antibodies in which a portion of the heavy and/or light chain is identical with, or homologous to, corresponding sequences in murine antibodies, in particular the murine CDRs, while the remainder of the chain(s) is (are) identical with, or homologous to, corresponding sequences in human antibodies. Other embodiments of the disclosure include antigen-binding fragments of these monoclonal antibodies that exhibit binding and biological properties similar or identical to the monoclonal antibodies. The antibodies of the present disclosure can comprise kappa or lambda light chain constant regions, and heavy chain IgA, IgD, IgE, IgG, or IgM constant regions, including those of IgG subclasses IgG1, IgG2, IgG3, and IgG4 and in some cases with various mutations to alter Fc receptor function.

The monoclonal antibodies containing the presently disclosed murine CDRs can be prepared by any of the various methods known to those skilled in the art, including recombinant DNA methods.

Reviews of current methods for antibody engineering and improvement can be found, for example, in P. Chames, Ed., (2012) Antibody Engineering: Methods and Protocols, Second Edition (Methods in Molecular Biology, Book 907), Humana Press, ISBN-10: 1617799734; C. R. Wood, Ed., (2011) Antibody Drug Discovery (Molecular Medicine and Medicinal Chemistry, Book 4), Imperial College Press; R. Kontermann and S. Dubel, Eds., (2010) Antibody Engineering Volumes 1 and 2 (Springer Protocols), Second Edition; and W. Strohl and L. Strohl (2012) Therapeutic antibody engineering: Current and future advances driving the strongest growth area in the pharmaceutical industry, Woodhead Publishing.

Methods for producing and purifying antibodies and antigen-binding fragments are well known in the art and can be found, for example, in Harlow and Lane (1988) Antibodies, A Laboratory Manual, Cold Spring Harbor Laboratory Press, Cold Spring Harbor, N.Y., chapters 5-8 and 15.

A full-length antibody as it exists naturally is a “Y” shaped immunoglobulin (Ig) molecule comprising four polypeptide chains: two identical heavy (H) chains and two identical light (L) chains, interconnected by disulfide bonds. The amino terminal portion of each chain, termed the fragment antigen binding region (FAB), includes a variable region of about 100-110 or more amino acids primarily responsible for antigen recognition via the complementarity determining regions (CDRs) contained therein. The carboxy-terminal portion of each chain defines a constant region (the “Fc” region) primarily responsible for effector function.

The CDRs are interspersed with regions that are more conserved, termed frameworks (“FRs”). Amino acid sequences of many FRs are well known in the art. Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions are in accordance with the well-known Kabat numbering convention Kabat et al. (1991) Sequences of Proteins of Immunological Interest, Fifth Edition. NIH Publication No. 91-3242.

As described herein, the “antigen-binding site” can also be defined as the “Hypervariable regions”, “HVRs”, or “HVs”, and refer to the structurally hypervariable regions of antibody variable domains as defined by Chothia and Lesk (Chothia and Lesk, Mol. Biol. 196:901-917, 1987). There are six HVRs, three in VH (H1, H2, H3) and three in VL (L1, L2, L3). We used herein CDRs as defined by Kabat except in H-CDR1, which is extended to include H1.

There are five types of mammalian immunoglobulin (Ig) heavy chains, denoted by the Greek letters α (alpha), δ (delta), ε (epsilon), γ (gamma), and μ (mu), which define the class or isotype of an antibody as IgA, IgD, IgE, IgG, or IgM, respectively. IgG antibodies can be further divided into subclasses, for example, IgG1, IgG2, IgG3, and IgG4.

Each heavy chain type is characterized by a particular constant region with a sequence well known in the art. The constant region is identical in all antibodies of the same isotype, but differs in antibodies of different isotypes. Heavy chains γ, α, and δ have a constant region composed of three tandem immunoglobulin (Ig) domains, and a hinge region for added flexibility. Heavy chains μ and ε have a constant region composed of four Ig domains.

The hinge region is a flexible amino acid stretch that links the Fc and Fab portions of an antibody. This regions contains cysteine residues that can form disulfide bonds, connecting two heavy chains together.

The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, light chains are classified as kappa (κ) or lambda (λ), and are characterized by a particular constant region as known in the art. A light chain has two successive domains: one variable domain at the amino-terminal end, and one constant domain at the carboxy-terminal end. Each antibody contains two light chains that are always identical; only one type of light chain, κ or λ, is present per antibody in mammals.

The Fc region, composed of two heavy chains that contribute three or four constant domains depending on the class of the antibody, plays a role in modulating immune cell activity. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects, including opsonization, cell lysis, and degranulation of mast cells, basophils and eosinophils.

As used herein, the term “epitope” refers to a specific arrangement of amino acids located on a peptide or protein to which an antibody or antibody fragment binds. Epitopes often consist of a chemically active surface grouping of molecules such as amino acids or sugar side chains, and have specific three dimensional structural characteristics as well as specific charge characteristics. Epitopes can be linear, i.e., involving binding to a single sequence of amino acids, or conformational, i.e., involving binding to two or more sequences of amino acids in various regions of the antigen that may not necessarily be contiguous in the linear sequence.

As used herein, the terms “specifically binds”, “bind specifically”, “specific binding”, and the like as applied to the present antibody compounds refer to the ability of a specific binding agent (such as an antibody) to bind to a target molecular species in preference to binding to other molecular species with which the specific binding agent and target molecular species are admixed. A specific binding agent is said specifically to recognize a target molecular species when it can bind specifically to that target.

As used herein, the term “binding affinity” refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

As used herein, the term “sequence identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs

Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, Altschul, S. F. et al., J. Mol. Biol. 215: 403-410 (1990) and Altschul et al. Nucl. Acids Res. 25: 3389-3402 (1997).

One example of an algorithm that is suitable for determining percent sequence identity and sequence similarity is the BLAST algorithm, which is described in (Altschul, S., et al., NCBI NLM NIH Bethesda, Md. 20894; and Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when: the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

As used herein, the terms “humanized”, “humanization”, and the like, refer to grafting of the murine monoclonal antibody CDRs disclosed herein to human FRs and constant regions. Also encompassed by these terms are possible further modifications to the murine CDRs, and human 1-Rs, by the methods disclosed in, for example, Kashmiri et al. (2005) Methods 36(1):25-34 and Hou et al. (2008) J. Biochem. 144(1):115-120, respectively, to improve various antibody properties, as discussed below.

As used herein, the term “humanized antibodies” refers to mAbs and antigen binding fragments thereof, including the antibody compounds disclosed herein, that have binding and functional properties according to the disclosure similar to those disclosed herein, and that have 1-Rs and constant regions that are substantially human or fully human surrounding CDRs derived from a non-human antibody.

As used herein, the term “FR” or “framework sequence” refers to any one of FRs 1 to 4. Humanized antibodies and antigen binding fragments encompassed by the present disclosure include molecules wherein any one or more of FRs 1 to 4 is substantially or fully human, i.e., wherein any of the possible combinations of individual substantially or fully human FRs 1 to 4, is present. For example, this includes molecules in which FR1 and FR2, FR1 and FR3, FR1, FR2, and FR3, etc., are substantially or fully human. Substantially human frameworks are those that have at least 80% sequence identity to a known human germline framework sequence. Preferably, the substantially human frameworks have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity, to a framework sequence disclosed herein, or to a known human germline framework sequence.

Fully human frameworks are those that are identical to a known human germline framework sequence. Human FR germline sequences can be obtained from the international ImMunoGeneTics (IMGT) database and from The Immunoglobulin FactsBook by Marie-Paule Lefranc and Gerard Lefranc, Academic Press, 2001, the contents of which are herein incorporated by reference in their entirety.

The Immunoglobulin Facts Book is a compendium of the human germline immunoglobulin genes that are used to create the human antibody repertoire, and includes entries for 203 genes and 459 alleles, with a total of 837 displayed sequences. The individual entries comprise all the human immunoglobulin constant genes, and germline variable, diversity, and joining genes that have at least one functional or open reading frame allele, and which are localized in the three major loci. For example, germline light chain FRs can be selected from the group consisting of: IGKV3D-20, IGKV2-30, IGKV2-29, IGKV2-28, IGKV1-27, IGKV3-20, IGKV1-17, IGKV1-16, 1-6, IGKV1-5, IGKV1-12, IGKV1D-16, IGKV2D-28, IGKV2D-29, IGKV3-11, IGKV1-9, IGKV1-39, IGKV1D-39 and IGKV1D-33 and IGKJ1-5 and germline heavy chain FRs can be selected from the group consisting of: IGHV1-2, IGHV1-18, IGHV1-46, IGHV1-69, IGHV2-5, IGHV2-26, IGHV2-70, IGHV1-3, IGHV1-8, IGHV3-9, IGHV3-11, IGHV3-15, IGHV3-20, IGHV3-66, IGHV3-72, IGHV3-74, IGHV4-31, IGHV3-21, IGHV3-23, IGHV3-30, IGHV3-48, IGHV4-39, IGHV4-59 and IGHV5-51 and IGHJ1-6.

Substantially human FRs are those that have at least 80% sequence identity to a known human germline FR sequence. Preferably, the substantially human frameworks have at least 85%, at least 90%, at least 95%, at least 96%, at least 97%, at least 98%, or at least 99% sequence identity, to a framework sequences disclosed herein, or to a known human germline framework sequence.

CDRs encompassed by the present disclosure include not only those specifically disclosed herein, but also CDR sequences having sequence identities of at least 80%, at least 85%, at least 90%, at least 95%, at least 96%, at least 97%, at least 98%, or at least 99% sequence identity to a CDR sequence disclosed herein. Alternatively, CDRs encompassed by the present disclosure include not only those specifically disclosed herein, but also CDR sequences having 1, 2, 3, 4, or 5 amino acid changes at corresponding positions compared to CDR sequences disclosed herein. Such sequence identical, or amino acid modified, CDRs preferably bind to the antigen recognized by the intact antibody.

Humanized antibodies in addition to those disclosed herein exhibiting similar functional properties according to the present disclosure can be generated using several different methods Almagro et al. Frontiers in Biosciences. Humanization of antibodies. (2008) Jan. 1; 13:1619-33. In one approach, the parent antibody compound CDRs are grafted into a human framework that has a high sequence identity with the parent antibody compound framework. The sequence identity of the new framework will generally be at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identical to the sequence of the corresponding framework in the parent antibody compound. In the case of frameworks having fewer than 100 amino acid residues, one, two, three, four, five, six, seven, eight, nine, or ten amino acid residues can be changed. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful to generate humanized variants based on homology and back mutations include as described in Olimpieri et al. Bioinformatics. 2015 Feb. 1;31(3):434-435 and U.S. Pat. Nos. 4,816,397, 5,225,539, and 5,693,761; and the method of Winter and co-workers (Jones et al. (1986) Nature 321:522-525; Riechmann et al. (1988) Nature 332:323-327; and Verhoeyen et al. (1988) Science 239:1534-1536.

Humanization began with chimerization, a method developed during the first half of the 1980's (Morrison, S. L., M. J. Johnson, L. A. Herzenberg & V. T. Oi: Chimeric human antibody molecules: mouse antigen-binding domains with human constant region domains. Proc. Natl. Acad. Sci. USA., 81, 6851-5 (1984)), consisting of combining the variable (V) domains of murine antibodies with human constant (C) domains to generate molecules with ˜70% of human content.

Several different methods can be used to generate humanized antibodies, which are described herein. In one approach, the parent antibody compound CDRs are grafted into a human FR that has a high sequence identity with the parent antibody compound framework. The sequence identity of the new FR will generally be at least 80%, at least 85%, at least 90%, at least 95%, at least 96%, at least 97%, at least 98%, or at least 99% identical to the sequence of the corresponding FR in the parent antibody compound. In the case of FRs having fewer than 100 amino acid residues, one, two, three, four, five, or more amino acid residues can be changed. This grafting may result in a reduction in binding affinity compared to that of the parent antibody. If this is the case, the FR can be back-mutated to the parent framework at certain positions based on specific criteria disclosed by Queen et al. (1991) Proc. Natl. Acad. Sci. USA 88:2869. Additional references describing methods useful to generate humanized variants based on homology and back mutations include as described in Olimpieri et al. Bioinformatics. 2015 Feb. 1;31(3):434-435 and U.S. Pat. Nos. 4,816,397, 5,225,539, and 5,693,761; and the method of Winter and co-workers (Jones et al. (1986) Nature 321:522-525; Riechmann et al. (1988) Nature 332:323-327; and Verhoeyen et al. (1988) Science 239:1534-1536.

The identification of residues to consider for back-mutation can be carried out as described below. When an amino acid falls under the following category, the framework amino acid of the human germ-line sequence that is being used (the “acceptor FR”) is replaced by a framework amino acid from a framework of the parent antibody compound (the “donor FR”):

(a) the amino acid in the human FR of the acceptor framework is unusual for human frameworks at that position, whereas the corresponding amino acid in the donor immunoglobulin is typical for human frameworks at that position;

(b) the position of the amino acid is immediately adjacent to one of the CDRs; or

(c) any side chain atom of a framework amino acid is within about 5-6 angstroms (center-to-center) of any atom of a CDR amino acid in a three-dimensional immunoglobulin model.

When each of the amino acids in the human FR of the acceptor framework and a corresponding amino acid in the donor framework is generally unusual for human frameworks at that position, such amino acid can be replaced by an amino acid typical for human frameworks at that position. This back-mutation criterion enables one to recover the activity of the parent antibody compound.

Another approach to generating humanized antibodies exhibiting similar functional properties to the antibody compounds disclosed herein involves randomly mutating amino acids within the grafted CDRs without changing the framework, and screening the resultant molecules for binding affinity and other functional properties that are as good as, or better than, those of the parent antibody compounds. Single mutations can also be introduced at each amino acid position within each CDR, followed by assessing the effects of such mutations on binding affinity and other functional properties. Single mutations producing improved properties can be combined to assess their effects in combination with one another.

Further, a combination of both of the foregoing approaches is possible. After CDR grafting, one can back-mutate specific FRs in addition to introducing amino acid changes in the CDRs. This methodology is described in Wu et al. (1999) J. Mol. Biol. 294: 151-162.

Applying the teachings of the present disclosure, a person skilled in the art can use common techniques, e.g., site-directed mutagenesis, to substitute amino acids within the presently disclosed CDR and FR sequences and thereby generate further variable region amino acid sequences derived from the present sequences. Up to all naturally occurring amino acids can be introduced at a specific substitution site. The methods disclosed herein can then be used to screen these additional variable region amino acid sequences to identify sequences having the indicated in vivo functions. In this way, further sequences suitable for preparing humanized antibodies and antigen-binding portions thereof in accordance with the present disclosure can be identified. Preferably, amino acid substitution within the frameworks is restricted to one, two, three, four, or five positions within any one or more of the four light chain and/or heavy chain FRs disclosed herein. Preferably, amino acid substitution within the CDRs is restricted to one, two, three, four, or five positions within any one or more of the three light chain and/or heavy chain CDRs. Combinations of the various changes within these FRs and CDRs described above are also possible.

That the functional properties of the antibody compounds generated by introducing the amino acid modifications discussed above conform to those exhibited by the specific molecules disclosed herein can be confirmed by the methods in Examples disclosed herein.

As described above, to circumvent the problem of eliciting human anti-murine antibody (HAMA) response in patients, murine antibodies have been genetically manipulated to progressively replace their murine content with the amino acid residues present in their human counterparts by grafting their complementarity determining regions (CDRs) onto the variable light (V_{L}) and variable heavy (V_{H}) frameworks of human immunoglobulin molecules, while retaining those murine framework residues deemed essential for the integrity of the antigen-combining site. However, the xenogeneic CDRs of the humanized antibodies may evoke anti-idiotypic (anti-Id) response in patients.

To minimize the anti-Id response, a procedure to humanize xenogeneic antibodies by grafting onto the human frameworks only the CDR residues most crucial in the antibody-ligand interaction, called “SDR grafting”, has been developed, wherein only the crucial specificity determining residues (SDRs) of CDRS are grafted onto the human frameworks. This procedure, described in Kashmiri et al. (2005) Methods 36(1):25-34, involves identification of SDRs through the help of a database of the three-dimensional structures of the antigen-antibody complexes of known structures, or by mutational analysis of the antibody-combining site. An alternative approach to humanization involving retention of more CDR residues is based on grafting of the ‘abbreviated’ CDRs, the stretches of CDR residues that include all the SDRs. Kashmiri et al. also discloses a procedure to assess the reactivity of humanized antibodies to sera from patients who had been administered the murine antibody.

Another strategy for constructing human antibody variants with improved immunogenic properties is disclosed in Hou et al. (2008) J. Biochem. 144(1):115-120. These authors developed a humanized antibody from 4C8, a murine anti-human CD34 monoclonal antibody, by CDR grafting using a molecular model of 4C8 built by computer-assisted homology modelling. Using this molecular model, the authors identified FR residues of potential importance in antigen binding. A humanized version of 4C8 was generated by transferring these key murine FR residues onto a human antibody framework that was selected based on homology to the murine antibody FR, together with the murine CDR residues. The resulting humanized antibody was shown to possess antigen-binding affinity and specificity similar to that of the original murine antibody, suggesting that it might be an alternative to murine anti-CD34 antibodies routinely used clinically.

Embodiments of the present disclosure encompass antibodies created to avoid recognition by the human immune system containing CDRs disclosed herein in any combinatorial form such that contemplated mAbs can contain the set of CDRs from a single murine mAb disclosed herein, or light and heavy chains containing sets of CDRs comprising individual CDRs derived from two or three of the disclosed murine mAbs. Such mAbs can be created by standard techniques of molecular biology and screened for desired activities using assays described herein. In this way, the disclosure provides a “mix and match” approach to create novel mAbs comprising a mixture of CDRs from the disclosed murine mAbs to achieve new, or improved, therapeutic activities.

Monoclonal antibodies or antigen-binding fragments thereof encompassed by the present disclosure that “compete” with the molecules disclosed herein are those that bind human CD47 at site(s) that are identical to, or overlapping with, the site(s) at which the present molecules bind. Competing monoclonal antibodies or antigen-binding fragments thereof can be identified, for example, via an antibody competition assay. For example, a sample of purified or partially purified human CD47 extracellular domain can be bound to a solid support. Then, an antibody compound, or antigen binding fragment thereof, of the present disclosure and a monoclonal antibody or antigen-binding fragment thereof suspected of being able to compete with such disclosure antibody compound are added. One of the two molecules is labeled. If the labeled compound and the unlabeled compound bind to separate and discrete sites on CD47, the labeled compound will bind to the same level whether or not the suspected competing compound is present. However, if the sites of interaction are identical or overlapping, the unlabeled compound will compete, and the amount of labeled compound bound to the antigen will be lowered. If the unlabeled compound is present in excess, very little, if any, labeled compound will bind. For purposes of the present disclosure, competing monoclonal antibodies or antigen-binding fragments thereof are those that decrease the binding of the present antibody compounds to CD47 by about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, or about 99%. Details of procedures for carrying out such competition assays are well known in the art and can be found, for example, in Harlow and Lane (1988) Antibodies, A Laboratory Manual, Cold Spring Harbor Laboratory Press, Cold Spring Harbor, N.Y. Such assays can be made quantitative by using purified antibodies. A standard curve is established by titrating one antibody against itself, i.e., the same antibody is used for both the label and the competitor. The capacity of an unlabeled competing monoclonal antibody or antigen-binding fragment thereof to inhibit the binding of the labeled molecule to the plate is titrated. The results are plotted, and the concentrations necessary to achieve the desired degree of binding inhibition are compared.

Whether mAbs or antigen-binding fragments thereof that compete with antibody compounds of the present disclosure in such competition assays possess the same or similar functional properties of the present antibody compounds can be determined via these methods in conjunction with the methods described in Examples 3-5, below. In various embodiments, competing antibodies for use in the therapeutic methods encompassed herein possess biological activities as described herein in the range of from about 50% to about 100% or about 125%, or more, compared to that of the antibody compounds disclosed herein. In some embodiments, competing antibodies possess about 50%, about 60%, about 70%, about 80%, about 85%, about 90%, about 91%, about 92%, about 93%, about 94%, about 95%, about 96%, about 97%, about 98%, about 99%, or identical biological activity compared to that of the antibody compounds disclosed herein as determined by the methods disclosed in the Examples presented below.

The mAbs or antigen-binding fragments thereof, or competing antibodies useful in the compositions and methods can be any of the isotypes described herein. Furthermore, any of these isotypes can comprise further amino acid modifications as follows.

The monoclonal antibody or antigen-binding fragment thereof, or competing antibody described herein can be of the human IgG1 isotype.

The human IgG1 constant region of the monoclonal antibody, antigen-binding fragment thereof, or competing antibody described herein can be modified to alter antibody half-life. Antibody half-life is regulated in large part by Fc-dependent interactions with the neonatal Fc receptor (Roopenian and Alikesh, 2007). The human IgG1 constant region of the monoclonal antibody, antigen-binding fragment thereof, or competing antibody can be modified to increase half-life include, but are not limited to amino acid modifications N434A, T307A/E380A/N434A (Petkova et al., 2006, Yeung et al., 2009); M252Y/S254T/T256E (Dall'Acqua et al., 2006); T250Q/M428L (Hinton et al., 2006); and M428L/N434S (Zalevsky et al., 2010).

As opposed to increasing half-life, there are some circumstances where decreased half-life would be desired, such as to reduce the possibility of adverse events associated with high Antibody-Dependent Cellular Cytotoxicity (ADCC) and Complement-Dependent Cytotoxicity (CDC) antibodies (Presta 2008). The human IgG1 constant region of the monoclonal antibody, antigen-binding fragment thereof, or competing antibody described herein can be modified to decrease half-life and/or decrease endogenous IgG include, but are not limited to amino acid modifications I253A (Petkova et al., 2006); P2571/N434H, D376V/N434H (Datta-Mannan et al., 2007); and M252Y/S254T/T256E/H433K/N434F (Vaccaro et al., 2005).

The human IgG1 constant region of the monoclonal antibody, antigen-binding fragment thereof, or competing antibody described herein can be modified to increase or decrease antibody effector functions. These antibody effector functions include, but are not limited to, Antibody-Dependent Cellular Cytotoxicity (ADCC), Complement-Dependent Cytotoxicity (CDC), Antibody-Dependent Cellular Phagocytosis (ADCP), C 1 q binding, and altered binding to Fc receptors.

The human IgG1 constant region of the monoclonal antibody, antigen-binding fragment thereof, or competing antibody described herein can be modified to increase antibody effector function include, but are not limited to amino acid modifications S298A/E333A/K334 (Shields et al., 2001); S239D/I332E and S239D/A330L/1332E (Lazar et al., 2006); F234L/R292P/Y300L, F234L/R292P/Y300L/P393L, and F243L/R292P/Y300L/V3051/P396L (Stevenhagen et al., 2007); G236A, G236A/S239D/I332E, and G236A/S239D/A330L/I332E (Richards et al., 2008); K326A/E333A, K326A/E333S and K326W/E333S (Idusogie et al., 2001); S267E and S267E/L328F (Smith et al., 2012); H268F/S324T, S267E/H268F, S267E/S234T, and S267E/H268F/S324T (Moore et al., 2010); S298G/T299A (Sazinsky et al., 2008); E382V/M428I (Jung et al., 2010).

The human IgG1 constant region of the monoclonal antibody, antigen-binding fragment thereof, or competing antibody described herein can be modified to decrease antibody effector function include, but are not limited to amino acid modifications N297A and N297Q (Bolt et al., 1993, Walker et al., 1989); L234A/L235A (Xu et al., 2000); K214T/E233P/L234V/L235A/G236-deleted/A327G/P331A/D356E/L358M (Ghevaert et al., 2008); C226S/C229S/E233P/L234V/L235A (McEarchern et al., 2007); S267E/L328F (Chu et al., 2008).

The human IgG1 constant region of the monoclonal antibody, antigen-binding fragment thereof, or competing antibody described herein can be modified to decrease antibody effector function include, but are not limited to amino acid modifications V234A/G237A (Cole et al., 1999); E233D, G237D, P238D, H268Q, H268D, P271G, V309L, A330S, A330R, P331S, H268Q/A330S/V309L/P331S, H268D/A330S/V309L/P331S, H268Q/A330R/V309L/P331S, H268D/A330R/V309L/P331S, E233D/A330R, E233D/A330S, E233D/P271G/A330R, E233D/P271G/A330S, G237D/H268D/P271G, G237D/H268Q/P271G, G237D/P271G/A330R, G237D/P271G/A330S, E233D/H268D/P271G/A330R, E233D/H268Q/P271G/A330R, E233D/H268D/P271G/A330S, E233D/H268Q/P271G/A330S, G237D/H268D/P271G/A330R, G237D/H268Q/P271G/A330R, G237D/H268D/P271G/A330S, G237D/H268Q/P271G/A330S, E233D/G237D/H268D/P271G/A330R, E233D/G237D/H268Q/P271G/A330R, E233D/G237D/H268D/P271G/A330S, E233D/G237D/H268Q/P271G/A330S, P238D/E233D/A330R, P238D/E233D/A330S, P238D/E233D/P271G/A330R, P238D/E233D/P271G/A330S, P238D/G237D/H268D/P271G, P238D/G237D/H268Q/P271G, P238D/G237D/P271G/A330R, P238D/G237D/P271G/A330S, P238D/E233D/H268D/P271G/A330R, P238D/E233D/H268Q/P271G/A330R, P238D/E233D/H268D/P271G/A330S, P238D/E233D/H268Q/P271G/A330S, P238D/G237D/H268D/P271G/A330R, P238D/G237D/H268Q/P271G/A330R, P238D/G237D/H268D/P271G/A330S, P238D/G237D/H268Q/P271G/A330S, P238D/E233D/G237D/H268D/P271G/A330R, P238D/E233D/G237D/H268Q/P271G/A330R, P238D/E233D/G237D/H268D/P271G/A330S, P238D/E233D/G237D/H268Q/P271G/A330S (An et al., 2009, Mimoto, 2013).

The monoclonal antibody or antigen-binding fragment thereof, or competing antibody described herein can be of the human IgG2 isotype.

The human IgG2 constant region of the monoclonal antibody, antigen-binding fragment thereof, or competing antibody described herein can be modified to increase or decrease antibody effector functions. These antibody effector functions include, but are not limited to, Antibody-Dependent Cellular Cytotoxicity (ADCC), Complement-Dependent Cytotoxicity (CDC), Antibody-Dependent Cellular Phagocytosis (ADCP), and C1q binding, and altered binding to Fc receptors.

The human IgG2 constant region of the monoclonal antibody, antigen-binding fragment thereof, or competing antibody described herein can be modified to increase antibody effector function include, but are not limited to the amino acid modification K326A/E333S (Idusogie et al., 2001).

The human IgG2 constant region of the monoclonal antibody, antigen-binding fragment thereof, or competing antibody described herein can be modified to decrease antibody effector function include, but are not limited to amino acid modifications V234A/G237A (Cole et al., 1999); E233D, G237D, P238D, H268Q, H268D, P271G, V309L, A330S, A330R, P331S, H268Q/A330S/V309L/P331S, H268D/A330S/V309L/P331S, H268Q/A330R/V309L/P331S, H268D/A330R/V309L/P331S, E233D/A330R, E233D/A330S, E233D/P271G/A330R, E233D/P271G/A330S, G237D/H268D/P271G, G237D/H268Q/P271G, G237D/P271G/A330R, G237D/P271G/A330S, E233D/H268D/P271G/A330R, E233D/H268Q/P271G/A330R, E233D/H268D/P271G/A330S, E233D/H268Q/P271G/A330S, G237D/H268D/P271G/A330R, G237D/H268Q/P271G/A330R, G237D/H268D/P271G/A330S, G237D/H268Q/P271G/A330S, E233D/G237D/H268D/P271G/A330R, E233D/G237D/H268Q/P271G/A330R, E233D/G237D/H268D/P271G/A330S, E233D/G237D/H268Q/P271G/A330S, P238D/E233D/A330R, P238D/E233D/A330S, P238D/E233D/P271G/A330R, P238D/E233D/P271G/A330S, P238D/G237D/H268D/P271G, P238D/G237D/H268Q/P271G, P238D/G237D/P271G/A330R, P238D/G237D/P271G/A330S, P238D/E233D/H268D/P271G/A330R, P238D/E233D/H268Q/P271G/A330R, P238D/E233D/H268D/P271G/A330S, P238D/E233D/H268Q/P271G/A330S, P238D/G237D/H268D/P271G/A330R, P238D/G237D/H268Q/P271G/A330R, P238D/G237D/H268D/P271G/A330S, P238D/G237D/H268Q/P271G/A330S, P238D/E233D/G237D/H268D/P271G/A330R, P238D/E233D/G237D/H268Q/P271G/A330R, P238D/E233D/G237D/H268D/P271G/A330S, P238D/E233D/G237D/H268Q/P271G/A330S (An et al., 2009, Mimoto, 2013).

The Fc region of a human IgG2 of the monoclonal antibody, antigen-binding fragment thereof, or competing antibody described herein can be modified to alter isoform and/or agonistic activity, include, but are not limited to amino acid modifications C127S (C_{H1} domain), C232S, C233S, C232S/C233S, C236S, and C239S (White et al., 2015, Lightle et al., 2010).

The monoclonal antibody or antigen-binding fragment thereof, or competing antibody described herein can be of the human IgG3 isotype.

The human IgG3 constant region of the monoclonal antibody, or antigen binding fragment thereof, wherein said human IgG3 constant region of the monoclonal antibody, or antigen-binding fragment thereof can be modified at one or more amino acid(s) to increase antibody half-life, Antibody-Dependent Cellular Cytotoxicity (ADCC), Complement-Dependent Cytotoxicity (CDC), or apoptosis activity.

The human IgG3 constant region of the monoclonal antibody, or antigen-binding fragment thereof, wherein said human IgG3 constant region of the monoclonal antibody, or antigen-binding fragment thereof can be modified at amino acid R435H to increase antibody half-life.

The monoclonal antibody or antigen-binding fragment thereof, or competing antibody described herein can be of the human IgG4 isotype.

The human IgG4 constant region of the monoclonal antibody, antigen-binding fragment thereof, or competing antibody described herein can be modified to decrease antibody effector functions. These antibody effector functions include, but are not limited to, Antibody-Dependent Cellular Cytotoxicity (ADCC) and Antibody-Dependent Cellular Phagocytosis (ADCP).

The human IgG4 constant region of the monoclonal antibody, antigen-binding fragment thereof, or competing antibody described herein can be modified to prevent Fab arm exchange and/or decrease antibody effector function include, but are not limited to amino acid modifications F234A/L235A (Alegre et al., 1994); S228P, L235E and S228P/L235E (Reddy et al., 2000).

As used herein, the term “tumor” refers to all neoplastic cell growth and proliferation, whether malignant or benign, and all pre-cancerous and cancerous cells and tissues.

The terms “cancer”, “cancerous”, and “tumor” are not mutually exclusive as used herein.

The terms “cancer” and “cancerous” refer to or describe the physiological condition in mammals that is typically characterized by aberrant cell growth/proliferation. Examples of cancers include, but are not limited to, carcinoma, lymphoma (i.e., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “susceptible cancer” as used herein refers to a cancer, cells of which express CD47, and are responsive to treatment with an antibody or antigen binding fragment thereof, or competing antibody or antigen binding fragment thereof, of the present disclosure.

The term “autoimmune disease” as used herein refers to when the body's immune system turns against itself and mistakenly attacks healthy cells.

The term “inflammatory disease” as used herein refers to a disease characterized by inflammation which is a fundamental pathologic process consisting of a dynamic complex of histologically apparent cytologic changes, cellular infiltration, and mediator release that occurs in the affected blood vessels and adjacent tissues in response to an injury or abnormal stimulation caused by a physical, chemical, or biologic agent, including the local reactions and resulting morphologic changes; the destruction or removal of the injurious material; and the responses that lead to repair and healing.

The term “autoinflammatory disease” as used herein refers to a disease that results when the innate immune system causes inflammation for unknown reasons.

As used herein, the term “ischemia” refers to a vascular phenomenon in which a decrease in the blood supply to a bodily organ, tissue, or part is caused, for instance, by constriction or obstruction of one or more blood vessels. Ischemia sometimes results from vasoconstriction or thrombosis or embolism. Ischemia can lead to direct ischemic injury, tissue damage due to cell death caused by reduced oxygen supply. Ischemia can occur acutely, as during surgery, or from trauma to tissue incurred in accidents, injuries and war settings, or following harvest of organs intended for subsequent transplantation, for example. It can also occur sub-acutely, as found in atherosclerotic peripheral vascular disease, where progressive narrowing of blood vessels leads to inadequate blood flow to tissues and organs. When a tissue is subjected to ischemia, a sequence of chemical events is initiated that may ultimately lead to cellular dysfunction and necrosis. If ischemia is ended by the restoration of blood flow, a second series of injurious events ensue, producing additional injury. Thus, whenever there is a transient decrease or interruption of blood flow in a subject, the resultant injury involves two components—the direct injury occurring during the ischemic interval, and the indirect or reperfusion injury that follows.

“Ischemic stroke” can be caused by several different kinds of diseases. The most common problem is narrowing of the arteries in the neck or head. This is most often caused by atherosclerosis, or gradual cholesterol deposition. If the arteries become too narrow, blood cells may collect in them and form blood clots (thrombi). These blood clots can block the artery where they are formed (thrombosis), or can dislodge and become trapped in arteries closer to the brain (embolism). Cerebral stroke can occur when atherosclerotic plaque separates away partially from the vessel wall and occludes the flow of blood through the blood vessel.

As used herein, the term “Reperfusion” refers to restoration of blood flow to tissue that is ischemic, due to decrease in blood flow. Reperfusion is a procedure for treating infarction or other ischemia, by enabling viable ischemic tissue to recover, thus limiting further necrosis. However, reperfusion can itself further damage the ischemic tissue, causing reperfusion injury. In addition to the immediate injury that occurs during deprivation of blood flow, “ischemic/reperfusion injury” involves tissue injury that occurs after blood flow is restored. Current understanding is that much of this injury is caused by chemical products, free radicals, and active biological agents released by the ischemic tissues.

“Nitric oxide (NO) donor, precursor, or nitric oxide generating topical agent” refers to a compound or agent that either delivers NO, or that can be converted to NO through enzymatic or non-enzymatic processes. Examples include, but are not limited to, NO gas, isosorbide dinitrite, nitrite, nitroprusside, nitroglycerin, 3-Morpholinosydnonimine (SIN-1), S-nitroso-N-acetyl-penicillamine (SNAP), Diethylenetriamine/NO (DETA/NO), S-nitrosothiols, Bidil®, and arginine.

“Soluble guanylyl cyclase (sGC)” is the receptor for nitric oxide in vascular smooth muscle. In the cardiovascular system, nitric oxide is endogenously generated by endothelial nitric oxide synthase from L-arginine, and activates soluble guanylyl cyclase in adjacent vascular smooth muscle cells to increase cGMP levels, inducing vascular relaxation. Nitric oxide binds to the normally reduced heme moiety of soluble guanylyl cyclase, and increases the formation of cGMP from GTP, leading to a decrease in intracellular calcium, vasodilation, and anti-inflammatory effects. Oxidation of the heme iron on sGC decreases responsiveness of the enzyme to nitric oxide, and promotes vasoconstriction. The nitric oxide-sGC-cGMP pathway therefore plays an important role in cardiovascular diseases. Nitrogen-containing compounds such as sodium azide, sodium nitrite, hydroxylamine, nitroglycerin, and sodium nitroprusside have been shown to stimulate sGC, causing an increase in cGMP, and vascular relaxation. In contrast to stimulators of sGC, which bind to reduced sGC, activators of sGC activate the oxidized or heme-deficient sGC enzyme that is not responsive to nitric oxide, i.e., they stimulate sGC independent of redox state. While stimulators of of sGC can enhance the sensitivity of reduced sGC to nitric oxide, activators of sGC can increase sGC enzyme activity even when the enzyme is oxidized and is therefore less, or unresponsive, to nitric oxide. Thus, sGC activators are non-nitric oxide based. Note the reviews of Nossaman et al. (2012) Critical Care Research and Practice, Volume 2012, article 290805, and Derbyshire and Marletta (2012) Ann. Rev. Biochem. 81:533-559.

“An agent that activates soluble guanylyl cyclase” refers, for example, to organic nitrates (Artz et al. (2002) J. Biol. Chem. 277:18253-18256); protoporphyrin IX (Ignarro et al. (1982) Proc. Natl. Acad. Sci. USA 79:2870-2873); YC-1 (Ko et al. (1994) Blood 84:4226-4233); BAY 41-2272 and BAY 41-8543 (Stasch et al. (2001 Nature 410 (6825): 212-5), CMF-1571, and A-350619 (reviewed in Evgenov et al. (2006) Nat. Rev. Drug. Discov. 5:755-768); BAY 58-2667 (Cinaciguat; Frey et al. (2008) Journal of Clinical Pharmacology 48 (12): 1400-10); BAY 63-2521 (Riociguat; Mittendorf et al. (2009) Chemmedchem 4 (5): 853-65). Additional soluble guanylyl cyclase activators are disclosed in Stasch et al. (2011) Circulation 123:2263-2273; Derbyshire and Marletta (2012) Ann. Rev. Biochem. 81:533-559, and Nossaman et al. (2012) Critical Care Research and Practice, Volume 2012, Article ID 290805, pages 1-12.

cGMP can also be increased by inhibiting degradation using phosphodiesterase inhibitors. Examples of “an agent that inhibits cyclic nucleotide phosphodiesterases” include, tadalafil, vardenafil, udenafil, and sildenafil avanafil.

As used herein, term “treating” or “treat” or “treatment” means slowing, interrupting, arresting, controlling, stopping, reducing, or reversing the progression or severity of a sign, symptom, disorder, condition, or disease, but does not necessarily involve a total elimination of all disease-related signs, symptoms, conditions, or disorders. The term “treating” and the like refer to a therapeutic intervention that ameliorates a sign or symptom of a disease or pathological condition after it has begun to develop.

As used herein, term “effective amount” refers to the amount or dose of an antibody compound of the present disclosure which, upon single or multiple dose administration to a patient or organ, provides the desired treatment or prevention.

The precise effective amount for any particular subject will depend upon their size and health, the nature and extent of their condition, and the therapeutics or combination of therapeutics selected for administration. The effective amount for a given patient is determined by routine experimentation and is within the judgment of a clinician. Therapeutically effective amounts of the present antibody compounds can also comprise an amount in the range of from about 0.1 mg/kg to about 150 mg/kg, from about 0.1 mg/kg to about 100 mg/kg, from about 0.1 mg/kg to about 50 mg/kg, or from about 0.05 mg/kg to about 10 mg/kg per single dose administered to a harvested organ or to a patient. Known antibody-based pharmaceuticals provide guidance in this respect. For example, Herceptin™ is administered by intravenous infusion of a 21 mg/ml solution, with an initial loading dose of 4 mg/kg body weight and a weekly maintenance dose of 2 mg/kg body weight; Rituxan™ is administered weekly at 375 mg/m²; for example.

A therapeutically effective amount for any individual patient can be determined by the health care provider by monitoring the effect of the antibody compounds on tumor regression, circulating tumor cells, tumor stem cells or anti-tumor responses. Analysis of the data obtained by these methods permits modification of the treatment regimen during therapy so that optimal amounts of antibody compounds of the present disclosure, whether employed alone or in combination with one another, or in combination with another therapeutic agent, or both, are administered, and so that the duration of treatment can be determined as well. In this way, the dosing/treatment regimen can be modified over the course of therapy so that the lowest amounts of antibody compounds used alone or in combination that exhibit satisfactory efficacy are administered, and so that administration of such compounds is continued only so long as is necessary to successfully treat the patient. Known antibody-based pharmaceuticals provide guidance relating to frequency of administration e.g., whether a pharmaceutical should be delivered daily, weekly, monthly, etc. Frequency and dosage may also depend on the severity of symptoms.

In some embodiments antibody compounds of the present disclosure can be used as medicaments in human and veterinary medicine, administered by a variety of routes including, but not limited to, oral, intravenous, intramuscular, intra-arterial, intramedullary, intraperitoneal, intrathecal, intraventricular, transdermal, transcutaneous, topical, subcutaneous, intratumoral, intranasal, enteral, sublingual, intravaginal, intravesicular or rectal routes. The compositions can also be administered directly into a lesion such as a tumor. Dosage treatment may be a single dose schedule or a multiple dose schedule. Hypo sprays may also be used to administer the pharmaceutical compositions. Typically, the therapeutic compositions can be prepared as injectables, either as liquid solutions or suspensions. Solid forms suitable for solution in, or suspension in, liquid vehicles prior to injection can also be prepared. Veterinary applications include the treatment of companion/pet animals, such as cats and dogs; working animals, such as guide or service dogs, and horses; sport animals, such as horses and dogs; zoo animals, such as primates, cats such as lions and tigers, bears, etc.; and other valuable animals kept in captivity.

Such pharmaceutical compositions can be prepared by methods well known in the art. See, e.g., Remington: The Science and Practice of Pharmacy, 21^{st} Edition (2005), Lippincott Williams & Wilkins, Philadelphia, Pa., and comprise one or more antibody compounds disclosed herein, and a pharmaceutically acceptable, for example, physiologically acceptable, carrier, diluent, or excipient.

The present disclosure describes murine, chimeric, and humanized anti-CD47 mAbs with distinct functional profiles. These antibodies possess one or more distinct combinations of properties selected from the following: 1) exhibit cross-reactivity with one or more species homologs of CD47; 2) block the interaction between CD47 and its ligand SIRPα; 3) do not block the interaction between CD47 and its ligand SIRPα; 4) increase phagocytosis of human tumor cells, 4) induce death of susceptible human tumor cells; 5) do not induce cell death of human tumor cells; 5) reverse TSP1 inhibition of the nitric oxide (NO) pathway and/or 6) do not reverse TSP1 inhibition of the NO pathway.

The anti-CD47 antibodies and antigen binding fragments thereof of the present disclosure possess combinations of properties that are distinct from the anti-CD47 antibodies of the prior art. These properties and characteristics will now be described in further detail.

### Binding to CD47 of Different Species

The anti-CD47 antibodies, and antigen binding fragments thereof, of the present disclosure bind human CD47. In certain embodiments, the anti-CD47 antibodies exhibit cross-reactivity with one or more species homologs of CD47, for example CD47 homologs of non-human primate origin. In certain embodiments, the anti-CD47 antibodies and antigen binding fragments thereof of the present disclosure bind to human CD47 and to CD47 of non-human primate, mouse, rat, and/or rabbit origin. The cross-reactivity with other species homologs can be particularly advantageous in the development and testing of therapeutic antibodies. For example, pre-clinical toxicology testing of therapeutic antibodies is frequently carried out in non-human primate species including, but not limited to, cynomolgus monkey, green monkey, rhesus monkey and squirrel monkey. Cross-reactivity with these species homologs can therefore be particularly advantageous for the development of antibodies as clinical candidates.

### Blocking the Interaction Between CD47 and SIRPα and Promoting Phagocytosis

CD47, also known as integrin associated protein (IAP), is a 50 kDa cell surface receptor that is comprised of an extracellular N-terminal IgV domain, a five membrane spanning transmembrane domain, and a short C-terminal intracellular tail that is alternatively spliced.

Two ligands bind to CD47: Signal Regulatory Protein alpha (SIRPα) and Thrombospondin-1 (TSP1). TSP1 is present in plasma and synthesized by many cells, including platelets. SIRPα is expressed on hematopoietic cells, which include macrophages and dendritic cells.

When SIRPα on a phagocyte engages CD47 on a target cell, this interaction prevents phagocytosis of the target cell. The interaction of CD47 and SIRPα effectively sends a “don't eat me” signal to the phagocyte (Oldenborg et al. Science 288: 2051-2054, 2000). Blocking the interaction of SIRPα and CD47 with an anti-CD47 mAb in a therapeutic context can provide an effective anti-cancer treatment by promoting the uptake and clearance of cancer cells by the host's immune system. Thus, an important functional characteristic of some anti-CD47 mAbs is the ability to block the interaction of CD47 and SIRPα, resulting in phagocytosis of CD47 expressing tumor cells by macrophages. Several anti-CD47 mAbs have been shown to block the interaction of CD47 and SIRPα, including B6H12 (Seiffert et al. Blood 94:3633-3643,1999; Latour et al. J. Immunol. 167: 2547-2554, 2001; Subramanian et al. Blood 107: 2548-2556, 2006; Liu et al. J. Biol. Chem. 277: 10028-10036, 2002; Rebres et al et al. J. Cellular Physiol. 205: 182-193, 2005), BRIC126 (Vernon-Wilson et al. Eur J Immunol. 30: 2130-2137, 2000; Subramanian et al. Blood 107: 2548-2556, 2006), CC2C6 (Seiffert et al. Blood 94:3633-3643,1999), and 1F7 (Rebres et al. J. Cellular Physiol. 205: 182-193, 2005). B6H12 and BRIC126 have also been shown to cause phagocytosis of human tumor cells by human and mouse macrophages (Willingham et al. Proc Natl Acad Sci USA 109(17):6662-6667, 2012; Chao et al. Cell 142:699-713, 2012; EP 2 242 512 B1). Other existing anti-CD47 mAbs, such as 2D3, does not block the interaction of CD47 and SIRPα (Seiffert et al. Blood 94:3633-3643,1999; Latour et al. J. Immunol. 167: 2547-2554, 2001; Rebres et al. J. Cellular Physiol. 205: 182-193, 2005), and does not cause phagocytosis of tumor cells (Willingham et al. Proc Natl Acad Sci USA 109(17):6662-6667, 2012; Chao et al. Cell 142:699-713, 2012; EP 2 242 512 B1).

As used herein, the term “blocks SIRPα binding to human CD47” refers to a greater than 50% reduction of SIRPα-Fc binding to CD47 on Jurkat cells by an anti-CD47 mAb.

The anti-CD47 mAbs of the disclosure described herein, block the interaction of CD47 and SIRPα and increase phagocytosis of human tumor cells.

“Phagocytosis” of cancer cells refers to the engulfment and digestion of such cells by macrophages, and the eventual digestion or degradation of these cancer cells and the release of digested or degraded cellular components extracellularly, or intracellularly to undergo further processing. Anti-CD47 monoclonal antibodies that block SIRPα binding to CD47 increase macrophage phagocytosis of cancer cells. SIRPα binding to CD47 on cancer cells would otherwise allow these cells to escape macrophage phagocytosis. The cancer cell may be viable or living cancer cells.

### Inducing Death of Tumor Cells

Some soluble anti-CD47 mAbs initiate a cell death program on binding to CD47 on tumor cells, resulting in collapse of mitochrondrial membrane potential, loss of ATP generating capacity, increased cell surface expression of phosphatidylserine (detected by increased staining for annexin V) and cell death without the participation of caspases or fragmentation of DNA. Such soluble anti-CD47 mAbs have the potential to treat a variety of solid and hematological cancers. Several soluble anti-CD47 mAbs which have been shown to induce tumor cell death, including MABL-1, MABL-2 and fragments thereof (U.S. Pat. No. 8,101,719; Uno et al. Oncol Rep. 17: 1189-94, 2007; Kikuchi et al. Biochem Biophys Res. Commun. 315: 912-8, 2004), Ad22 (Pettersen et al. J. Immuno. 166: 4931-4942, 2001; Lamy et al. J. Biol. Chem. 278: 23915-23921, 2003), and 1F7 (Manna et al. J. Immunol. 170: 3544-3553, 2003; Manna et al. Cancer Research, 64: 1026-1036, 2004). Some of the anti-CD47 mAbs of the disclosure described herein induce cell death of human tumor cells.

The terms “inducing cell death” or “kills” and the like, are used interchangeably herein to mean that addition of an antibody compound of the present disclosure to cultured cancer cells causes these cells to display quantifiable characteristics associated with cell death including any one, or more, of the following:
1. Increased binding of Annexin V (in the presence of calcium ion) to the tumor cells as detected by flow cytometry or confocal fluorescence microscopy;
2. Increased uptake of the fluorescent compound propidium iodide (as assayed by flow cytometry) or 7-aminoactinomycin D (7-AAD as assayed by flow cytometry) or trypan blue (scored with light microscopy) by the tumor cells
3. Loss of mitochondrial function and membrane potential by the tumor cells as assayed by one of several available measures (potentiometric fluorescent dyes such as DiO-C6 or JC1 or formazan-based assays such as MTT or WST-1).

Induction of cell death refers to the ability of certain of the soluble anti-CD47 antibodies, murine antibodies, chimeric antibodies, humanized antibodies, or antigen-binding fragments thereof (and competing antibodies and antigen-binding fragments thereof) disclosed herein to kill cancer cells via a cell autonomous mechanism without participation of complement or other cells including, but not limited to, T cells, neutrophils, natural killer cells, macrophages, or dendritic cells. Quantifiably, induction of cell death includes, but is not limited to, a greater than 2-fold increase in annexin V staining of human tumor cells caused by soluble anti-CD47 mAb compared to the background obtained with the negative control antibody (humanized, isotype-matched antibody).

Among the present murine, chimeric or humanized mAbs, those that induce cell death of human tumor cells cause increased Annexin V binding similar to the findings reported for anti-CD47 mAbs Ad22 (Pettersen et al. J. Immuno. 166: 4931-4942, 2001; Lamy et al. J. Biol. Chem. 278: 23915-23921, 2003); 1F7 (Manna and Frazier J. Immunol. 170:3544-3553, 2003; Manna and Frazier Cancer Res. 64:1026-1036, 2004); and MABL-1 and 2 (U.S. Pat. No. 7,531,643 B2; U.S. Pat. No. 7,696,325 B2; U.S. Pat. No. 8,101,719 B2).

Cell viability assays are described in NCI/NIH guidance manual that describes numerous types of cell based assays that can be used to assess induction of cell death caused by CD47 antibodies: “Cell Viability Assays”, Terry L Riss, PhD, Richard A Moravec, BS, Andrew L Niles, MS, Helene A Benink, PhD, Tracy J Worzella, MS, and Lisa Minor, PhD. Contributor Information, published May 1, 2013.

### Modulation of the NO Pathway

As noted above, TSP1 is also a ligand for CD47. The TSP1/CD47 pathway opposes the beneficial effects of the NO pathway in many cell types, including, but not limited to, vascular cells. The NO pathway consists of any of three enzymes (nitric oxide synthases, NOS I, NOS II and NOS III) that generate bioactive gas NO using arginine as a substrate. NO can act within the cell in which it is produced, or in neighboring cells, to activate the enzyme soluble guanylyl cyclase that produces the messenger molecule cyclic GMP (cGMP). The proper functioning of the NO/cGMP pathway is essential for protecting the cardiovascular system against stresses including, but not limited to, those resulting from wounding, inflammation, hypertension, metabolic syndrome, ischemia, and IRI. In the context of these cellular stresses the inhibition of the NO/cGMP pathway by the TSP1/CD47 system exacerbates the effects of stress. This is a particular problem in the cardiovascular system where both cGMP and cAMP play important protective roles. There are many cases in which ischemia and reperfusion injury cause or contribute to disease, trauma, and poor outcomes of surgical procedures.

As disclosed herein, one of more of the chimeric or humanized anti-CD47 antibodies will reverse TSP1 inhibition of cGMP production. Reversal will be complete (>80%) or intermediate (20%-80%). This reversal of TSP1 inhibition of cGMP production will demonstrate that the anti-CD47 mAbs have the ability to increase NO signaling and suggest utility in protecting the cardiovascular system against stresses including, but not limited to, those resulting from wounding, inflammation, hypertension, metabolic syndrome, ischemia, and ischemia-reperfusion injury (IRI). Additional assay systems, for example smooth muscle cell contraction, will also be expected to show that some of the chimeric or humanized antibodies reverse the inhibitory actions of TSP1 on downstream effects resulting from the activation of NO signaling.

As disclosed herein, “complete reversal of NO pathway inhibition” refers to greater than 80% reversal of TSP1 inhibition of NO signaling by an anti-CD47 mAb compared to a negative control, humanized isotype-matched antibody.

As disclosed herein, “intermediate reversal of NO pathway inhibition” refers to 20-80% reversal of TSP1 inhibition of NO signaling by an anti-CD47 mAb compared to a negative control, humanized isotype-matched antibody.

As disclosed herein, “no reversal of NO pathway inhibition” refers to less than 20% reversal of TSP1 inhibition of NO signaling by an anti-CD47 mAb compared to a negative control, humanized isotype-matched antibody.

### Preferred Combinations of Functional Properties

Anti-CD47 mAbs exist in the prior art with combinations of some, but not all, of the functional characteristics described herein. Previously, it has been shown that humanized anti-CD47 mAbs such as AB6.12 IgG1, AB6.12-IgG4P, and AB6.12-IgG4PE (U.S. Pat. No. 9,045,541, US Patent Publication 2014/0161799, WO Publication 2014/093678, US Patent Publication 2014/0363442) and 5F9 (Mounho-Zamora B. et al. The Toxicologist, Supplement to Toxicological Sciences, 2015; 144 (1): Abstract 596: 127, Liu et al. PLoS One. 2015 Sep. 21; 10(9): e0137345) bind human CD47, block the interaction of CD47 and SIRPα and cause phagocytosis of human tumor cells. The humanized CD47 mAbs AB6.12 IgG1, AB6.12-IgG4P, and AB6.12-IgG4PE also do not cause hemagglutination of human RBCs (U.S. Pat. No. 9,045,541). The 5F9 humanized anti-CD47 mAb binds to and causes hemagglutination of human RBCs (Uger R. et al. Cancer Res 2014; 74(19 Suppl): Abstract nr 5011, Sikic B. et al. J Clin Oncol 2016;34 (suppl; abstract 3019). Murine anti-CD47 mAbs B6H12, BRIC126, and CC2C6 block the interaction of CD47 and SIRPα, cause phagocytosis, and bind to and cause hemagglutination of human RBCs (Petrova P. et al. Cancer Res 2015; 75(15 Suppl): Abstract nr 4271, Seiffert et al. Blood 94:3633-3643,1999; Vernon-Wilson et al. Eur J Immunol. 30: 2130-2137, 2000; Latour et al. J. Immunol. 167: 2547-2554, 2001; Subramanian et al. Blood 107: 2548-2556, 2006; Liu et al. J Biol. Chem. 277: 10028-10036, 2002). Murine anti-CD47 mAbs MABL-1 and MABL-2 bind to human CD47, induce tumor cell death and cause RBC hemagglutination (U.S. Pat. No. 8,101,719); murine mAb Ad22 binds to human CD47 and induces tumor cell death (Pettersen et al. J. Immunol. 166: 4931-4942, 2001; Lamy et al. J Biol Chem. 278: 23915-23921, 2003); and murine mAb 1F7 binds to human CD47, blocks the interaction of CD47 and SIRPα and induces tumor cell death (Rebres et al. J. Cellular Physiol. 205: 182-193, 2005; Manna et al. J. Immunol. 170: 3544-3553, 2003; Manna et al. Cancer Research, 64: 1026-1036, 2004).

In another preferred embodiment described herein, the monoclonal antibody, or antigen binding fragment thereof also specifically binds to non-human primate CD47, wherein non-human primate may include, but is not limited to, cynomolgus monkey, green monkey, rhesus monkey and squirrel monkey.

In yet another preferred embodiment described herein, the monoclonal antibody, or antigen binding fragment thereof binds human, non-human primate, mouse, rabbit, and rat CD47.

Described herein, are murine, chimeric, and humanized anti-CD47 mAbs with distinct functional profiles. These antibodies possess distinct combinations of properties selected from the following: 1) exhibit cross-reactivity with one or more species homologs of CD47; 2) block the interaction between CD47 and its ligand SIRPα; 3) do not block the interaction between CD47 and its ligand SIRPα; 4) increase phagocytosis of human tumor cells, 4) induce death of susceptible human tumor cells; 5) do not induce cell death of human tumor cells; 5) reverse TSP1 inhibition of the nitric oxide (NO) pathway and/or 6) do not reverse TSP1 inhibition of the NO pathway

### CD47 Antibodies

Many human cancers up-regulate cell surface expression of CD47 and those expressing the highest levels of CD47 appear to be the most aggressive and the most lethal for patients. Increased CD47 expression is thought to protect cancer cells from phagocytic clearance by sending a “don't eat me” signal to macrophages via SIRPα, an inhibitory receptor that prevents phagocytosis of CD47-bearing cells (Oldenborg et al. Science 288: 2051-2054, 2000; Jaiswal et al. (2009) Cell 138(2):271-851; Chao et al. (2010) Science Translational Medicine 2(63):63ra94). Thus, the increase of CD47 expression by many cancers provides them with a cloak of “selfness” that slows their phagocytic clearance by macrophages and dendritic cells.

Antibodies that block CD47 and prevent its binding to SIRPα have shown efficacy in human tumor in murine (xenograft) tumor models. Such blocking anti-CD47 mAbs exhibiting this property increase the phagocytosis of cancer cells by macrophages, which can reduce tumor burden (Majeti et al. (2009) Cell 138 (2): 286-99; U.S. Pat. No. 9,045,541; Willingham et al. (2012) Proc Natl Acad. Sci. USA 109(17):6662-6667; Xiao et al. (2015) Cancer Letters 360:302-309; Chao et al. (2012) Cell 142:699-713; Kim et al. (2012) Leukemia 26:2538-2545) and may ultimately lead to generation of an adaptive immune response to the tumor (Tseng et al. (2013) PNAS 110 (27):11103-11108; Soto-Pantoja et al. (2014) Cancer Res. 74 (23): 6771-6783; Liu et al. (2015) Nat. Med. 21 (10): 1209-1215).

However, there are mechanisms by which anti-CD47 mAbs can attack transformed cells that have not yet been exploited in the treatment of cancer. Multiple groups have shown that particular anti-human CD47 mAbs induce cell death of human tumor cells. Anti-CD47 mAb Ad22 induces cell death of multiple human tumor cells lines (Pettersen et al. J. Immuno. 166: 4931-4942, 2001; Lamy et al. J. Biol. Chem. 278: 23915-23921, 2003). AD22 was shown to indice rapid mitochondrial dysfunction and rapid cell death with early phosphatidylserine exposure and a drop in mitochondrial membrane potential (Lamy et al. J. Biol. Chem. 278: 23915-23921, 2003). Anti-CD47 mAb MABL-2 and fragments thereof induce cell death of human leukemia cell lines, but not normal cells in vitro and had an anti-tumor effect in in vivo xenograft models. (Uno et al. (2007) Oncol. Rep. 17 (5): 1189-94). Anti-human CD47 mAb 1F7 induces cell death of human T cell leukemias (Manna and Frazier (2003) J. Immunol. 170: 3544-53) and several breast cancers (Manna and Frazier (2004) Cancer Research 64 (3):1026-36). 1F7 kills CD47-bearing tumor cells without the action of complement or cell mediated killing by NK cells, T cells, or macrophages. Instead, anti-CD47 mAb 1F7 acts via a non-apoptotic mechanism that involves a direct CD47-dependent attack on mitochondria, discharging their membrane potential and destroying the ATP-generating capacity of the cell leading to rapid cell death. It is noteworthy that anti-CD47 mAb 1F7 does not kill resting leukocytes, which also express CD47, but only those cells that are “activated” by transformation. Thus, normal circulating cells, many of which express CD47, are spared while cancer cells are selectively killed by the tumor-toxic CD47 mAb (Manna and Frazier (2003) J. Immunol. 170: 3544-53). This mechanism can be thought of as a proactive, selective and direct attack on tumor cells in contrast to the passive mechanism of causing phagocytosis by simply blocking CD47/SIRPα binding. Importantly, mAb 1F7 also blocks binding of SIRPα to CD47 (Rebres et al et al. J. Cellular Physiol. 205: 182-193, 2005) and thus it can act via two mechanisms: (1) direct tumor toxicity, and (2) causing phagocytosis of cancer cells. A single mAb that can accomplish both functions may be superior to one that only blocks CD47/SIRPα binding.

Following periods of tissue ischemia, the initiation of blood flow causes damage referred to as “ischemia-reperfusion injury” or IRI. IRI contributes to poor outcomes in many surgical procedures where IRI occurs due to the necessity to stop blood flow for a period of time, in many forms/causes of trauma in which blood flow is interrupted and later restored by therapeutic intervention and in procedures required for organ transplantation, cardio/pulmonary bypass procedures, reattachment of severed body parts, reconstructive and cosmetic surgeries and other situations involving stopping and restarting blood flow. Ischemia itself causes many physiological changes that, by themselves would eventually lead to cell and tissue necrosis and death. Reperfusion poses its own set of damaging events including generation of reactive oxygen species, thrombosis, inflammation and cytokine mediated damage. The pathways that are limited by the TSP1-CD47 system are precisely those that would be of most benefit in combating the damage of IRI, including the NO pathway. Thus, blocking the TSP1-CD47 pathway, as with the antibodies disclosed herein, will provide more robust functioning of these endogenous protective pathways. Anti-CD47 mAbs have been shown to reduce organ damage in rodent models of renal warm ishchemia (Rogers et al. J Am Soc Nephrol. 23: 1538-1550, 2012), liver ischemia-reperfusion injury (Isenberg et al. Surgery. 144: 752-761, 2008), renal transplantation (Lin et al. Transplantation. 98: 394-401, 2014; Rogers et al. Kidney Interantional. 90: 334-347, 2016)) and liver transplantation, including steatotic livers (Xiao et al. Liver Transpl. 21: 468-477, 2015; Xiao et al. Transplantation. 100: 1480-1489, 2016). In addition, anti-CD47 mAb caused significant reductions of right ventricular systolic pressure and right ventricular hypertrophy in the monocrotaline model of pulmonary arterial hypertension (Bauer et al. Cardiovasc Res. 93: 682-693, 2012). Studies in skin flap models have shown that modulation of CD47, including with anti-CD47 mAbs, inhibits TSP1-mediated CD47 signaling. This results in inceased activity of the NO pathway, resulting in reduced IRI (Maxhimer et al. Plast Reconstr Surg. 124: 1880-1889, 2009; Isenberg et al. Arterioscler Throm Vasc Biol. 27: 2582-2588, 2007; Isenberg et al. Curr Drug Targets. 9: 833-841, 2008; Isenberg et al. Ann Surg. 247: 180-190, 2008)

Anti-CD47 mAbs have also been shown to be efficacious in models of other cardiovascular diseases. In the mouse transverse aortic constriction model of pressure overload left ventricular heart failure, anti-CD47 mAb mitigated cardiac myocyte hypertrophy, decreased left ventricular fibrosis, prevented an increase in left ventricular weight, decreased ventricular stiffness, and normalized changes in the pressure volume loop profile (Sharifi-Sanjani et al. J Am Heart Assoc., 2014). An anti-CD47 mAb ameliorated atherosclerosis in multiple mouse models (Kojima et al. Nature., 2016).

### Cancer Indications

Presently disclosed are anti-CD47 mAbs and antigen binding fragments thereof effective as cancer therapeutics which can be administered to patients, preferably parenterally, with susceptible hematologic cancers and solid tumors including, but not limited to, leukemias, including systemic mastocytosis, acute lymphocytic (lymphoblastic) leukemia (ALL), T cell-ALL, acute myeloid leukemia (AML), myelogenous leukemia, chronic lymphocytic leukemia (CLL), chronic myeloid leukemia (CML), myeloproliferative disorder/neoplasm, monocytic cell leukemia, and plasma cell leukemia; multiple myeloma (MM); Waldenstrom's Macroglobulinemia; lymphomas, including histiocytic lymphoma and T cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL; solid tumors, including ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, hepatocellular carcinoma (liver cancer, hepatoma), gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, meduloblastoma, astrocytoma, glioma, meningioma, gastrinoma, neuroblastoma, myelodysplastic syndrome, and sarcomas including, but not limited to, osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma; and melanoma.

### Treatment of Cancer

As is well known to those of ordinary skill in the art, combination therapies are often employed in cancer treatment as single-agent therapies or procedures may not be sufficient to treat or cure the disease or condition. Conventional cancer treatments often involve surgery, radiation treatment, the administration of a combination of cytotoxic drugs to achieve additive or synergistic effects, and combinations of any or all of these approaches. Especially useful chemotherapeutic and biologic therapy combinations employ drugs that work via different mechanisms of action, increasing cancer cell control or killing, increasing the ability of the immune system to control cancer cell growth, reducing the likelihood of drug resistance during therapy, and minimizing possible overlapping toxicities by permitting the use of reduced doses of individual drugs.

Classes of conventional anti-tumor/anti-neoplastic agents useful in the combination therapies encompassed by the present methods are disclosed, for example, in Goodman & Gilman's The Pharmacological Basis of Therapeutics, Twelfth Edition (2010) L. L. Brunton, B. A. Chabner, and B. C. Knollmann Eds., Section VIII, “Chemotherapy of Neoplastic Diseases”, Chapters 60-63, pp. 1665-1770, McGraw-Hill, NY, and include, for example, alkylating agents, antimetabolites, natural products, a variety of miscellaneous agents, hormones and antagonists, targeted drugs, monoclonal antibodies and other protein therapeutics.

In addition to the foregoing, the methods of the present disclosure are related to treatment of cancer indications and further comprises treating the patient via surgery, radiation, and/or administering to a patient in need thereof an effective amount of a chemical small molecule or biologic drug including, but not limited to, a peptide, polypeptide, protein, nucleic acid therapeutic, conventionally used or currently being developed, to treat tumorous conditions. This includes antibodies and antigen-binding fragments, other than those disclosed herein, cytokines, antisense oligonucleotides, siRNAs, and miRNAs.

The therapeutic methods disclosed and claimed herein include the use of the antibodies disclosed herein alone, and/or in combinations with one another, and/or with antigen-binding fragments thereof of the present disclosure that bind to CD47, and/or with competing antibodies exhibiting appropriate biological/therapeutic activity, as well, for example, all possible combinations of these antibody compounds to achieve the greatest treatment efficacy.

In addition, the present therapeutic methods also encompass the use of these antibodies, antigen-binding fragments thereof, competing antibodies and combinations thereof further in combination with: (1) any one or more anti-tumor therapeutic treatments selected from surgery, radiation, anti-tumor, anti-neoplastic agents, and combinations of any of these, or (2) any one or more of anti-tumor biological agents, or (3) equivalents of any of the foregoing of (1) or (2) as would be apparent to one of ordinary skill in the art, in appropriate combination(s) to achieve the desired therapeutic treatment effect for the particular indication.

Antibody and small molecule drugs that increase the immune response to cancer by modulating co-stimulatory or inhibitory interactions that influence the T cell response to tumor antigens, including inhibitors of immune checkpoints and modulators of co-stimulatory molecules, are also of particular interest in the context of the combination therapeutic methods encompassed herein and include, but are not limited to, other anti-CD47 antibodies. Administration of therapeutic agents that bind to the CD47 protein, for example, antibodies or small molecules that bind to CD47 and prevent interaction between CD47 and SIRPα, are administered to a patient, causing the clearance of cancer cells via phagocytosis. The therapeutic agent that binds to the CD47 protein is combined with a therapeutic agent such as an antibody, a chemical small molecule or biologic drug disclosed herein, directed against one or more additional cellular targets of CD70 (Cluster of Differentiation 70), CD200 (OX-2 membrane glycoprotein, Cluster of Differentiation 200), CD154 (Cluster of Differentiation 154, CD40L, CD40 ligand, Cluster of Differentiation 40 ligand), CD223 (Lymphocyte-activation gene 3, LAG3, Cluster of Differentiation 223), KIR (Killer-cell immunoglobulin-like receptors), GITR (TNFRSF18, glucocorticoid-induced TNFR-related protein, activation-inducible TNFR family receptor, AITR, Tumor necrosis factor receptor superfamily member 18), CD28 (Cluster of Differentiation 28), CD40 (Cluster of Differentiation 40, Bp50, CDW40, TNFRSFS, Tumor necrosis factor receptor superfamily member 5, p50), CD86 (B7-2, Cluster of Differentiation 86), CD160 (Cluster of Differentiation 160, BY55, NK1, NK28), CD258 (LIGHT, Cluster of Differentiation 258, Tumor necrosis factor ligand superfamily member 14, TNFSF14, HVEML, HVEM ligand, herpesvirus entry mediator ligand, LTg), CD270 (HVEM, Tumor necrosis factor receptor superfamily member 14, herpesvirus entry mediator, Cluster of Differentiation 270, LIGHTR, HVEA), CD275 (ICOSL, ICOS ligand, Inducible T-cell co-stimulator ligand, Cluster of Differentiation 275), CD276 (B7-H3, B7 homolog 3, Cluster of Differentiation 276), OX40L (OX40 Ligand), B7-H4 (B7 homolog 4, VTCN1, V-set domain-containing T-cell activation inhibitor 1), GITRL (Glucocorticoid-induced tumor necrosis factor receptor-ligand, glucocorticoid-induced TNFR-ligand), 4-1BBL (4-1BB ligand), CD3 (Cluster of Differentiation 3, T3D), CD25 (IL2Ra, Cluster of Differentiation 25, Interleukin-2 Receptor a chain, IL-2 Receptor a chain), CD48 (Cluster of Differentiation 48, B-lymphocyte activation marker, BLAST-1, signaling lymphocytic activation molecule 2, SLAMF2), CD66a (Ceacam-1, Carcinoembryonic antigen-related cell adhesion molecule 1, biliary glycoprotein, BGP, BGP1, BGPI, Cluster of Differentiation 66a), CD80 (B7-1, Cluster of Differentiation 80), CD94 (Cluster of Differentiation 94), NKG2A (Natural killer group 2A, killer cell lectin-like receptor subfamily D member 1, KLRD1), CD96 (Cluster of Differentiation 96, TActILE, T cell activation increased late expression), CD112 (PVRL2, nectin, Poliovirus receptor-related 2, herpesvirus entry mediator B, HVEB, nectin-2, Cluster of Differentiation 112), CD115 (CSF1R, Colony stimulating factor 1 receptor, macrophage colony-stimulating factor receptor, M-CSFR, Cluster of Differentiation 115), CD205 (DEC-205, LY75, Lymphocyte antigen 75, Cluster of Differentiation 205), CD226 (DNAM1, Cluster of Differentiation 226, DNAX Accessory Molecule-1, PTA1, platelet and T cell activation antigen 1), CD244 (Cluster of Differentiation 244, Natural killer cell receptor 2B4), CD262 (DRS, TrailR2, TRAIL-R2, Tumor necrosis factor receptor superfamily member 10b, TNFRSF10B, Cluster of Differentiation 262, KILLER, TRICK2, TRICKB, ZTNFR9, TRICK2A, TRICK2B), CD284 (Toll-like Receptor-4, TLR4, Cluster of Differentiation 284), CD288 (Toll-like Receptor-8, TLR8, Cluster of Differentiation 288), TNFSF15 (Tumor necrosis factor superfamily member 15, Vascular endothelial growth inhibitor, VEGI, TL1A), TDO2 (Tryptophan 2,3-dioxygenase, TPH2, TRPO), IGF-1R (Type 1 Insulin-like Growth Factor), GD2 (Disialoganglioside 2), TMIGD2 (Transmembrane and immunoglobulin domain-containing protein 2), RGMB (RGM domain family, member B), VISTA (V-domain immunoglobulin-containing suppressor of T-cell activation, B7-H5, B7 homolog 5), BTNL2 (Butyrophilin-like protein 2), Btn (Butyrophilin family), TIGIT (T cell Immunoreceptor with Ig and ITIM domains, Vstm3, WUCAM), Siglecs (Sialic acid binding Ig-like lectins), Neurophilin, VEGFR (Vascular endothelial growth factor receptor), ILT family (LIRs, immunoglobulin-like transcript family, leukocyte immunoglobulin-like receptors), NKG families (Natural killer group families, C-type lectin transmembrane receptors), MICA (MHC class I polypeptide-related sequence A), TGFβ (Transforming growth factor β), STING pathway (Stimulator of interferon gene pathway), Arginase (Arginine amidinase, canavanase, L-arginase, arginine transamidinase), EGFRvIII (Epidermal growth factor receptor variant III), and HHLA2 (B7-H7, B7y, HERV-H LTR-associating protein 2, B7 homolog 7), inhibitors of PD-1 (Programmed cell death protein 1, PD-1, CD279, Cluster of Differentiation 279), PD-L1 (B7-H1, B7 homolog 1, Programmed death-ligand 1, CD274, cluster of Differentiation 274), PD-L2 (B7-DC, Programmed cell death 1 ligand 2, PDCD1LG2, CD273, Cluster of Differentiation 273), CTLA-4 (Cytotoxic T-lymphocyte-associated protein 4, CD152, Cluster of Differentiation 152), BTLA (B- and T-lymphocyte attenuator, CD272, Cluster of Differentiation 272), Indoleamine 2,3-dioxygenase (IDO, IDO1), TIM3 (HAVCR2, Hepatitis A virus cellular receptor 2, T cell immunoglobulin mucin-3, KIM-3, Kidney injury molecule 3, TIMD-3, T cell immunoglobulin mucin-domain 3), A2A adenosine receptor (ADO receptor), CD39 (ectonucleoside triphosphate diphosphohydrolase-1, Cluster of Differentiation 39, ENTPD1), and CD73 (Ecto-5′-nucleotidase, 5′-nucleotidase, 5′-NT, Cluster of Differentiation 73), CD27 (Cluster of Differentiation 27), ICOS (CD278, Cluster of Differentiation 278, Inducible T-cell Co-stimulator), CD137 (4-1BB, Cluster of Differentiation 137, tumor necrosis factor receptor superfamily member 9, TNFRSF9), OX40 (CD134, Cluster of Differentiation 134), and TNFSF25 (Tumor necrosis factor receptor superfamily member 25), including antibodies, small molecules, and agonists, are also specifically contemplated herein. Additional agents include IL-10 (Interleukin-10, human cytokine synthesis inhibitory factor, CSIF) and Galectins.

YERVOY® (ipilimumab; Bristol-Meyers Squibb) is an example of an approved anti-CTLA-4 antibody.

KEYTRUDA® (pembrolizumab; Merck) and OPDIVO® (nivolumab; Bristol-Meyers Squibb Company) are examples of approved anti-PD-1 antibodies.

TECENTRIQ™ (atezolizumab; Roche) is an example of an approved anti-PD-L1 antibody.

BAVENCIO™ (avelumab; Merck KGaA and Pfizer and Eli Lilly and Company) is an example of an approved anti-PD-L1 antibody.

IMFINZI™ (Durvalumab; Medimmune/AstraZeneca) is an example of an approved anti-274 antibody.

### Ischemia-Reperfusion Injury (IRI)-Related, Autoimmune, Autoinflammatory, Inflammatory, and Cardiovascular Conditions and Diseases

Administration of a CD47 mAb or antigen binding fragment thereof disclosed herein can be used to treat a number of diseases and conditions in which IRI is a contributing feature, and to treat various autoimmune, autoinflammatory, inflammatory and cardiovascular diseases. These include: organ transplantation in which a mAb or antigen binding fragment thereof of the present invention is administered to the donor prior to organ harvest, to the harvested donor organ in the organ preservation solution, to the recipient patient, or to any combination thereof; skin grafting; surgical resections or tissue reconstruction in which such mAb or fragment is administered either locally by injection to the affected tissue or parenterally to the patient; reattachment of body parts; treatment of traumatic injury; pulmonary hypertension; pulmonary arterial hypertension; sickle cell disease (crisis); myocardial infarction; cerebrovascular disease; stroke; surgically-induced ischemia; acute kidney disease/kidney failure; any other condition in which IRI occurs and contributes to the pathogenesis of disease; autoimmune and inflammatory diseases, including arthritis, rheumatoid arthritis, multiple sclerosis, psoriasis, psoriatic arthritis, Crohn's disease, inflammatory bowel disease, ulcerative colitis, lupus, systemic lupus erythematous, juvenile rheumatoid arthritis, juvenile idiopathic arthritis, Grave's disease, Hashimoto's thyroiditis, Addison's disease, celiac disease, dermatomyositis, multiple sclerosis, myasthenia gravis, pernicious anemia, Sjogren syndrome, type I diabetes, vasculitis, uveitis and ankylosing spondylitis; autoinflammatory diseases, including familial Mediterrean fever, neonatal onset multisystem inflammatory disease, tumor necrosis factor (TNF) receptor-associated periodic syndrome, deficiency of the interleukin-1 receptor antagonist, Behcet's disease; cardiovascular diseases, including coronary heart disease, coronary artery disease, atherosclerosis, myocardial infarction, heart failure, and left ventricular heart failure.

Anti-CD47 mAbs and antigen binding fragments thereof of the present invention can also be used to increase tissue perfusion in a subject in need of such treatment. Such subjects can be identified by diagnostic procedures indicating a need for increased tissue perfusion. In addition, the need for increased tissue perfusion may arise because the subject has had, is having, or will have, a surgery selected from integument surgery, soft tissue surgery, composite tissue surgery, skin graft surgery, resection of a solid organ, organ transplant surgery, or reattachment or an appendage or other body part.

### Treatment of Ischemia-Reperfusion Injury (IRI)-Related Indications

The methods of the present disclosure, for example those related to treatment of IRI-related indications, can further comprise administering to a patient in need thereof an effective amount of therapeutic agent that binds to the CD47 protein and a nitric oxide donor, precursor, or both; a nitric oxide generating topical agent; an agent that activates soluble guanylyl cyclase; an agent that inhibits cyclic nucleotide phosphodiesterases; or any combination of any of the foregoing.

In these methods, the nitric oxide donor or precursor can be selected from NO gas, isosorbide dinitrate, nitrite, nitroprusside, nitroglycerin, 3-Morpholinosydnonimine (SIN-1), S-nitroso-N-acetylpenicillamine (SNAP), Diethylenetriamine/NO (DETA/NO), S-nitrosothiols, Bidil®, and arginine.

The agent that activates soluble guanylyl cyclase can be a non-NO (nitric oxide)-based chemical activator of soluble guanylyl cyclase that increases cGMP levels in vascular cells. Such agents bind soluble guanylyl cyclase in a region other than the NO binding motif, and activate the enzyme regardless of local NO or reactive oxygen stress (ROS). Non-limiting examples of chemical activators of soluble guanylyl cyclase include organic nitrates (Artz et al. (2002) J. Biol. Chem. 277:18253-18256); protoporphyrin IX (Ignarro et al. (1982) Proc. Natl. Acad. Sci. USA 79:2870-2873); YC-1 (Ko et al. (1994) Blood 84:4226-4233); BAY 41-2272 and BAY 41-8543 (Stasch et al. (2001 Nature 410 (6825): 212-5), CMF-1571, and A-350619 (reviewed in Evgenov et al. (2006) Nat. Rev. Drug. Discov. 5:755-768); BAY 58-2667 (Cinaciguat; Frey et al. (2008) Journal of Clinical Pharmacology 48 (12): 1400-10); BAY 63-2521 (Riociguat; Mittendorf et al. (2009) Chemmedchem 4 (5): 853-65). Additional soluble guanylyl cyclase activators are disclosed in Stasch et al. (2011) Circulation 123:2263-2273; Derbyshire and Marletta (2012) Ann. Rev. Biochem. 81:533-559, and Nossaman et al. (2012) Critical Care Research and Practice, Volume 2012, Article ID 290805, pages 1-12.

The agent that inhibits cyclic nucleotide phosphodiesterases can be selected from, tadalafil, vardenafil, udenafil, sildenafil and avanafil.

### Treatment of Autoimmune, Autoinflammatory, Inflammatory Diseases, and Cardiovascular Diseases

A therapeutic agent that binds to the CD47 protein for the treatment of an autoimmune, autoinflammatory, inflammatory disease and/or cardiovascular disease can be combined with one or more therapeutic agent(s) such as an antibody, a chemical small molecule, or biologic or a medical or surgical procedure which include, but are not limited to the following. For the treatment of autoimmune, autoinflammatory and inflammatory diseases, the combined therapeutic agents are: hydroxychloroquine, leflunomide, methotrexate, minocycline, sulfasalazine, abatacept, rituximab, tocilizumab, anti-TNF inhibitors or blockers (adalimumab, etanercept, infliximab, certolizumab pegol, golimumab), non-steroidal anti-inflammatory drugs, glucocorticoids, corticosteroids, intravenous immunoglobulin, anakinra, canakinumab, rilonacept, cyclophosphamide, mycophenolate mofetil, azathioprine, 6-mercaptopurine, belimumab, beta interferons, glatiramer acetate, dimethyl fumarate, fingolimod, teriflunomide, natalizumab, 5-aminosalicylic acid, mesalamine, cyclosporine, tacrolimus, pimecrolimus, vedolizumab, ustekinumab, secukinumab, ixekizumab, apremilast, budesonide and tofacitinib. For the treatment of atherosclerosis, the combined therapeutic agents or procedures are: medical procedures and/or surgery, including percutaneous coronary intervention (coronary angioplasty and stenting), coronary artery bypass grafting, and carotid endarterectomy; therapeutic agents, including angiotensin-converting enzyme (ACE) inhibitors (including ramipril, quinapril, captopril, and enalapril), calcium channel blockers (including amiodipine, nifedipine, verapamil, felodipine and diltiazem), angiotensin-receptor blockers (including eposartan, olmesarten, azilsartan, valsartan, telmisartan, losartan, candesartan, and irbesartan), the combination of ezetimibe and simvastatin, PCSK9 inhibitors (including alirocumab and evolocumab), anacetrapib, and HMG-CoA inhibitors (including atorvastatin, pravastatin, simvastatin, rosuvastatin, pitavastatin, lovastatin and fluvastatin). For the treatment of heart failure, the combined therapeutic agents are: ACE inhibitors, angiotensin receptor blockers, angiotensin receptor neprilsyn inhibitors (including the combination of sacubitril and valsartan), diuretics, digoxin, inotropes, beta blockers and aldosterone antagonists. For the treatment of pumonary hypertension the combined therapeutic agents are: sildenafil, tadalafil, ambrisentan, bosentan, macitentan, riociguat, treprostinil, epoprostenol, iloprost, and selexipag.

As disclosed herein, the anti-CD47 mAb is administered before, at the same time or after the combined therapeutic agents or medical or surgical procedures.

Another useful class of compounds for the combination therapies contemplated herein includes modulators of SIRPoc/CD47 binding such as antibodies to SIRPα, as well as soluble protein fragments of this ligand, or CD47 itself, inhibiting binding of, or interfering with binding of, SIRPα to CD47. It should be noted that the therapeutic methods encompassed herein include the use of the antibodies disclosed herein alone, in combination with one another, and/or with antigen-binding fragments thereof as well, for example, all possible combinations of these antibody compounds.

The examples illustrate various embodiments of the present disclosure, but should not be considered as limiting the disclosure to only these particularly disclosed embodiments.

### Diagnostics for CD47 Expression

Diagnostics (including complementary and companion) have been an area of focus in the field of oncology. A number of diagnostic assays have been developed for targeted therapeutics such as Herceptin (Genentech), Tarceva (OSI Pharmaceuticals/Genentech), Iressa (Astra Zeneca), and Erbitux (Imclone/Bristol Myers Squibb). The anti-CD47 mAbs antibodies of the disclosure are particularly well-suited to use in diagnostic applications. Accordingly, the disclosure provides a method to measure CD47 expression in tumor and/or immune cells, using an anti-CD47 mAb of the disclosure.

The anti-CD47 mAbs of the disclosure may be used in a diagnostic assay and/or in vitro method to measure CD47 expression in tumor and/or immune cells present in a patient's tumor sample. In particular, the anti-CD47 mAbs of the disclosure may bind CD47 on approximately 1% or more of tumor and/or immune cells present in a patient's sample as compared to a reference level. In another embodiment, the anti-CD47 mAbs may bind CD47 on approximately 5% or more of tumor and/or immune cells in a patient's sample as compared to a reference level, for example, or binding at least 10%, or at least 20%, or at least 30%, or at least about 40%, or at least about 50%, or at least about 60%, or at least about 70%, or at least about 80%, or at least about 90%, or between 10-100% as compared to a reference level. In yet another embodiment, the anti-CD47 mAbs may bind CD47 on tumor and/or immune cells in a patient's sample to at least about a 2-fold increase as compared to a reference level, or at least about 3-fold, or at least about a 4-fold, or at least about a 5-fold or at least about a 10-fold increase, or between 2-fold and 10-fold or greater as compared to a reference level. As described herein, the measurement of CD47 expression in a patient's sample provides biological and/or clinical information that enables decision making about the development and use of a potential drug therapy, notably the use of anti-CD47 antibodies for treating solid and hematological cancers, autoimmune disease, inflammatory disease, atherosclerosis, heart failure, in which the CD47 receptor plays a role.

In one embodiment, the in vitro method comprises, obtaining a patient sample, contacting the patient sample with a monoclonal antibody, or antigen-binding fragment thereof, which specifically binds to an epitope, and assaying for binding of the antibody to the patient sample, wherein binding of the antibody to the patient sample is diagnostic of CD47 expression in a patient sample.

Accordingly, a diagnostic assay in accordance with the disclosure may comprise contacting tumor and/or immune cells in a patient's sample with an anti-CD47 mAb, or an antigen binding fragment thereof, and assaying for binding of the anti-CD47 mAb to a patient's tumor sample, wherein binding of the anti-CD47 mAb to the patient sample is diagnostic of CD47 expression. Preferably, the patient's sample is a sample containing tumor cells. In this case, binding of the anti-CD47 mAb of the disclosure, or antigen binding fragment thereof, to the tumor cells may be assessed for CD47 expression. The levels of CD47 expression by tumor cells and/or immune cells of a patient's tumor sample may be predictive of clinical outcome in a patient.

Increased binding of anti-CD47 mAbs binding to cells in a patient's sample is associated with increased CD47 expression. In one embodiment, the anti-CD47 mAbs of the disclosure may bind to approximately 5% or more of tumor cells in a patient's sample and this may indicate that the patient would benefit from rapid intervention to a solid and hematological cancer. A diagnostic assay of this sort may be used to determine suitable therapeutic regimes for solid and hematological cancers with relatively high binding of anti-CD47 mAbs of the disclosure, i.e., increased CD47 expression.

It will be appreciated that the diagnostic assay disclosed herein has a number of advantages. The most important of these advantages is that the diagnostic assay of the disclosure may allow the user a greater deal of confidence in the CD47 expression in tumor and/or immune cells. The increased sensitivity of the diagnostic assay of the disclosure allows detection of CD47 in a patient's sample at lower levels than has previously been the case.

The anti-CD47 mAbs of the disclosure may be used as a diagnostic assay in relation to many forms of cancer. Particular forms of cancer that may advantageously be investigated for CD47 expression include susceptible hematologic cancers and solid tumors including, but not limited to, leukemias, lymphomas, and solid tumors.

The diagnostic assays of the disclosure may utilize any suitable means for detecting binding of an anti-CD47 mAb to measure CD47 expression. Suitable methods may be selected with reference to the nature of any reporter moiety used to label the anti-CD47 mAbs of the disclosure. Suitable techniques include, but are by no means limited to, flow cytometry, and enzyme linked immunosorbent assays (ELISA) and assays utilizing nanoparticles. It is particularly preferred that a diagnostic assay of the invention be one involving immunohistochemistry in which a tumor sample is exposed to an anti-CD47 mAb of the disclosure, and the level of cell labelling is assessed by immunohistochemistry.

### EXAMPLES

### Example 1

Amino Acid Sequences
Light Chain and Heavy Chain CDRs

| LCDR1 | | LCDR2 | | LCDR3 | |
|---|---|---|---|---|---|
| SEQ ID NO: 1 | RSSQSLVHSNGNTYLH | SEQ ID NO: 7 | KVSNRLS | SEQ ID NO: 11 | SQTTHVPYT |
| SEQ ID NO: 2 | RSSQSLENSNGDTYLN | SEQ ID NO: 8 | RVSNRFS | SEQ ID NO: 12 | LQVSHVPWT |
| SEQ ID NO: 1 | RSAQSLVHSNGNTYLH | SEQ ID NO: 9 | KVSNRFS | SEQ ID NO: 13 | SQSTHVPRT |
| SEQ ID NO: 1 | RSAQSLVHSNGNTYLH | SEQ ID NO: 9 | KVSNRFS | SEQ ID NO: 14 | SQSTHVLT |
| SEQ ID NO: 4 | RSSQNIVQSNGNTYLE | SEQ ID NO: 9 | KVFHRFS | SEQ ID NO: 15 | FQGSHVPWT |
| SEQ ID NO: 4 | RSSQNIVQSNGNTYLE | SEQ ID NO: 9 | KVFHRFS | SEQ ID NO: 16 | FQGSYVPWT |
| SEQ ID NO: 5 | RASSSIFYVD | SEQ ID NO: 10 | DTSKLAS | SEQ ID NO: 17 | QQWSSNPPT |
| SEQ ID NO: 6 | SASSSIFYVD | SEQ ID NO: 10 | DTSKLAS | SEQ ID NO: 17 | QQWSSNPPT |
| HCDR1 | | HCDR2 | | HCDR3 | |
| SEQ ID NO: 18 | GYTFTNYGMN | SEQ ID NO: 24 | WININTGEPTYAEDFKG | SEQ ID NO: 31 | WARGGNFDL |
| SEQ ID NO: 19 | GYTFTNYWIH | SEQ ID NO: 25 | YIDPNTVYTDYNQRFED | SEQ ID NO: 32 | GGKRGVDS |
| SEQ ID NO: 20 | GYTFTNYFLY | SEQ ID NO: 26 | DINPNAGSTNLNERFKS | SEQ ID NO: 33 | GGYTMDY |
| SEQ ID NO: 20 | GYTFTNYFLY | SEQ ID NO: 26 | DINPNAGSTNLNERFKS | SEQ ID NO: 33 | GGYTMDY |
| SEQ ID NO: 21 | DYTFTNYYIH | SEQ ID NO: 27 | WIYPGNNNNKYNEKFKG | SEQ ID NO: 33 | GGYTMDY |
| SEQ ID NO: 22 | GYTFTNYWMH | SEQ ID NO: 28 | YIDPRTAYTEYNQKFKD | SEQ ID NO: 35 | GGRVGLGY |
| SEQ ID NO: 22 | GYTFTNYWMH | SEQ ID NO: 29 | YIDPRTDYSEYNQKFKD | SEQ ID NO: 35 | GGRVGLGY |
| SEQ ID NO: 23 | GYSFTGYYMH | SEQ ID NO: 30 | RANPYNGGTSYNQKFKG | SEQ ID NO: 36 | NYGGSDAMDY |
| SEQ ID NO: 23 | GYSFTGYYMH | SEQ ID NO: 30 | RANPYNGGTSYNQKFKG | SEQ ID NO: 37 | NYGSSDAMDY |

Murine Light Chain (LC) and Murine Heavy Chain (HC) Variable Domains

| | | |
|---|---|---|
| SEQ ID NO: 38 | Vx14 LC | DVVLTQTPLSLPVGLGDQASISCRSSQSLVHSNGNTYLHWYLQKPGQSPKLLIYK |
| | | VSNRLSGVPDRFSGSGSGTDFTLRISRVEAEDLGVYFCSQTTHVPYTFGGGTELE |
| | | IK |
| SEQ ID NO: 39 | Vx10 LC | DVVMTQTPLSLPVSLGDQASISCRSSQSLENSNGDTYLNWYLQKPGQSPQLLIYR |
| | | VSNRFSGVLDRFSGSGSGTDFTLQISRVEAEDLGVYFCLQVSHVPWTFGGGTNLE |
| | | IK |
| SEQ ID NO: 40 | Vx11 LC | DVVMTQTPLSLPVSLGDQASISCRSSQSLENSNGDTYLNWYLQKPGQSPQLLIYR |
| | | VSNRFSGVLDRFSGSGSGTDFTLQISRVEAEDLGVYFCLQVSHVPWTFGGGTNLE |
| | | IK |
| SEQ ID NO: 41 | Vx12 LC | DVVMTQIPLSLPVSLGDQASISCRSSQSLVHSNGNTYLHWYLQKPGQSPKLLIYK |
| | | VSNRESGVPDRFSGSGSGTDFTLKISRVEAEDLGVYFCSQSTHVPRTFGGGTKLE |
| | | IK |
| SEQ ID NO: 42 | Vx13 LC | DVLMTQTPLSLPVSLGDQASISCRSSQSLVHSNGNTYLHWYLQKPGQSPNLLIYK |
| | | VSNRESGVPDRFSGSGSGTDFTLKINRVETEDLGIYFCSQSTHVLTFGAG |
| SEQ ID NO: 43 | Vx14 LC | DVLMTQTPLSLPVSLGDQASISCRSSQNIVQSNGNTYLEWYLQKPGQSPKLLIYK |
| | | VFHRFSGVPDRFSGSGSGTDFTLKISGVEAEDLGVYYCFQGSHVPWTFGGGTRLE |
| | | IK |
| SEQ ID NO: 44 | Vx16 LC | DVLMTQTPLSLPISLGDQASISCRSSQNIVQSNGNTYLEWYLQKPGQSPKLLIYK |
| | | VFHRFSGVPDRFSGSGSGTDFTLKISRVEAEDLGVYYCFQGSYVPWTFGGGTRLE |
| | | IK |
| SEQ ID NO: 45 | Vx17 LC | QIVLTQSPAIMSASPGERVTMTCRASSSIFYVDWYQQKSGTSPKRWIYDTSKLAS |
| | | GVPARFSGSGSGTSYSLTISSMEAEDAATYHCQQWSSNPPTFGAGTKLELK |
| SEQ ID NO: 46 | Vx18 LC | QIVLTQSPAIMSASPGERVTMTCSASSSIFYVDWYQQKSGTSPKRWIYDTSKLAS |
| | | GVPARFSGSGSGTSYSLTISSMEAEDAATYHCQQWSSNPPTFGAGTKLELK |
| SEQ ID NO: 47 | Vx14 LC | QIQLVQSGPHLKKPGETAKISCKASGYTFTNYGMNWVKQAPGKDLKWMGWININT |
| | | GEPTYAEDFKGRFVFSLETSAGTAYLQISNLKNEDTATYFCARWARGGNFDLWGQ |
| | | GTTLTVSS |
| SEQ ID NO: 48 | Vx10 LC | QVQLQQSGAELAKPGASVKMSCKASGYTFTNYWIHWIKQRPGQGLEWIGYIDPNT |
| | | VYTDYNQRHEDKATLTADKSSNTAYMQLNSLTSEDSAVYYCSRGGKRGVDSWGQG |
| | | TSVTVSS |
| SEQ ID NO: 49 | Vx11 LC | EVQLQQSGAQLVKPGTSMKLSCKASGYTFTNYFLYWVKQRPGQGLEWIGDINPNA |
| | | GSTNLNERFKSKATLTVDKSSSTAYLQLSGLTSEDSAVYYCTRGGYTMDYWGQGT |
| | | SVTVSS |
| SEQ ID NO: 50 | Vx12 HC | QVQLLQSGAQLVKPGTSMKLSCKASGYTFTNYFLYWVKQRPGQGLEWIGDINPNA |
| | | GSTNLNERFKSKATLTVDKSSSTAYLQLSGLTSEDSAVYYCTRGGYTMDYWGQGT |
| | | SVTVSS |
| SEQ ID NO: 51 | Vx13 HC | EVQLQQSGPEVVKPGASVRISCKASDYTETNYYIHWVRQRPGQGLEWIGWIYPGN |
| | | NNNKYNEKFKGKATLTEDTSSSTAYMQLSSLTSEDSAVYFCARGGYTMDYWGQG |
| SEQ ID NO: 52 | Vx15 HC | QVQLQQSGAELAKPGASVQMSCKASGYTFTNYWMHWVKQRSGQGLEWIGYIDPRT |
| | | AYTEYNQKFKDKATLTADKSSSTAYMRLSSLTSEDSAVYYCVGGGRVGLGYWGHG |
| | | SSVTVSS |
| SEQ ID NO: 53 | Vx16 HC | EVQLQQSGAELAKPGASVKMSCKASGYTFTNYWMHWVKQRPGQGLEWIGYIDPRT |
| | | DYSEYNQKFKDKATLTADKSSSTAYMQLSSLTSEDSAVYECAGGGRVGLGYWGHG |
| | | SSVTVSS |
| SEQ ID NO: 54 | Vx17 HC | EVQLQQSGPDLVKPGASVKISCKASGYSETGYYMHWVKQSHGKSLEWIGRANPYN |
| | | GGTSYNQKFKGKAILTVDKSSSTAYMELRSLTSEDSAVYYCARNYGGSDAMDYWG |
| | | QGTSITVAS |
| SEQ ID NO: 55 | Vx18 HC | EVQLQQSGPDLVKPGASVKISCKASGYSFTGYYMHWVKQSHGKSLEWIGRANPYN |
| | | GGTSYNQKFKGKAILTVDKSSSTAYMELRSLTSEDSAVYYCARNYGSSDAMDYWG |
| | | QGTSITVAS |

Chimeric Heavy Chain (HC) and Chimeric Light Chain (LC)

| | | |
|---|---|---|
| SEQ ID NO: 56 | Vx10_mh_L01 | DVVMTQTPLSLPVSLGDQASISCRSSQSLHNSNGDTYLNWYLQKPGQSPQLLIYR |
| | | VSNRFSGVLDRFSGSGSGTDFTLQISRVEAEDLGVYFCLQVSHVPWTFGGGTNLE |
| | | IKRTVAAPSVFIFPPSDEQLKSGTASVVCLLNNFYPREAKVQWKVDNALQSGNSQ |
| | | HSVTHQDSKDSTYSLSSTLTLSKADYHKHKVYACFVTHQGLSSPVTKSFNRGEC |
| SEQ ID NO: 57 | Vx10_mh_HC01 | QVQLQQSGAELAKPGASVKMSCKASGYTFTNYWIHWIKQRPGQGLEWIGYIDPNT |
| | | VYTDYNQRFEDKATLTADKSSNTAYMQLNSLTSEDSAVYYCSRGGKRGVDSWGQG |
| | | TSVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDVSHEDPEVKF |
| | | NWYVDGVEVHNAKTKPREEQYQSTYRVVSVLTVLHQDWLNGKEYKCKVSNKALPA |
| | | PIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |
| SEQ ID NO: 58 | Vx10_mh_HC02 | QVQLQQSGAELAKPGASVKMSCKASGYTFTNYWIHWIKQRPGQGLEWIGYIDPNT |
| | | VYTDYNQRFEDKATLTADKSSNTAYMQLNSLTSEDSAVYYCSRGGKRGVDSWGQG |
| | | TSVTVSSASTKGPSVFPLAPCSRSTSESTAALGCLVKDYFPEPVTVSWNSGALTS |
| | | GVHTFPAVLQSSGLYSLSSVVTVPSSSLGTKTYTCNVDHKPSNTKVDKRVESKYG |
| | | PPCPPCPAPEFEGGPSVFLFPPKPKDTLMISRTPEVTCVVVDVSQEDPEVQFNWY |
| | | VDGVEVHNAKTKPREEQFNSTYRVVSVLTVLHQDWLNGKEYKCKVSNKGLPSSIE |
| | | KTISKAKGQPREPQVYTLPPSQEEMTKNQVSLTCLVKGFYPSDIAVEWESNGQPE |
| | | NNYKTTPPVLDSDGSFFLYSRLTVDKSRWQEGNVFSCSVMHEALHNHYTQKSLSL |
| | | SLG |
| SEQ ID NO: 59 | Vx12_mh_LC01 | DVVMTQIPLSLPVSLGDQASISCRSSQSLVHSNGNTYLHWYLQKPGQSPKLLIYK |
| | | VSNRFSGVPDRFSGSGSGTDFTLKISRVEAEDLGVYFCSQSTHVPRTFGGGTKLE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 60 | Vx12_mh_HC01 | QVQLLQSGAQLVKPGTSMKLSCKASGYTFTNYFLYWVKQRPGQGLEWIGDINPNA |
| | | GSTNLNERFKSKATLTVDKSSSTAYLQLSGLTSEDSAVYYCTRGGYTMDYWGQGT |
| | | SVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFN |
| | | WYVDGVEVHNAKTKPREEQYQSTYRVVSVLTVLHQDWLNGKEYKCKVSNKALPAP |
| | | IEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |
| SEQ ID NO: 61 | Vx12_mh_HC02 | QVQLLQSGAQLVKPGTSMKLSCKASGYTFTNYFLYWVKQRPGQGLEWIGDINPNA |
| | | GSTNLNERFKSKATLTVDKSSSTAYLQLSGLTSEDSAVYYCTRGGYTMDYWGQGT |
| | | SVTVSSASTKGPSVFPLAPCSRSTSESTAALGCLVKDYFPEPVTVSWNSGALTSG |
| | | VHTFPAVLQSSGLYSLSSVVTVPSSSLGTKTYTCNVDHKPSNTKVDKRVESKYGP |
| | | PCPPCPAPEFEGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSIEK |
| | | TISKAKGQPREPQVYTLPPSQEEMTKNQVSLTCLVKGFYPSDIAVEWESNGQPEN |
| | | NYKTTPPVLDSDGSFFLYSRLTVDKSRWQEGNVFSCSVMHEALHNHYTQKSLSLS |
| | | LG |
| SEQ ID NO: 62 | Vx14_mh_LC01 | DVVLTQTPLSLPVGLGDQASISCRSSQSLVHSNGNTYLHWYLQKPGQSPKLLIYK |
| | | VSNRLSGVPDRFSGSGSGTDFTLRISRVEAEDLGVYFCSQTTHVPYTFGGGTELE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 63 | Vx14_mh_HC01 | QIQLVQSGPELKKPGETAKISCKASGYTFTNYGMNWVKQAPGKDLKWMGWININT |
| | | GEPTYAEDFKGRFVFSLETSAGTAYLQISNLKNEDTATYFCARWARGGNFDLWGQ |
| | | GTTLTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVVDVSHEDPEVK |
| | | FNWYVDGVEVHNAKTKPREEQYQSTYRVVSVLTVLHQDWLNGKEYKCKVSNKALP |
| | | APIEKTISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHNHYTQK |
| | | SLSLSPGK |
| SEQ ID NO: 64 | Vx14_mh_HC02 | QIQLVQSGPELKKPGETAKISCKASGYTFTNYGMNWVKQAPGKDLKWMGWININT |
| | | GEPTYAEDFKGRFVFSLETSAGTAYLQISNLKNEDTATYFCARWARGGNFDLWGQ |
| | | GTTLTVSSASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKVDKRVESKY |
| | | GPPCPPCPAPEFEGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNGQP |
| | | ENNYKTTPPVLDSDGSFFLYSRLTVDKSRWQEGNVFSCSVMHEALHNHYTQKSLS |
| | | LSLG |

Human Light Chain (LC) Variable Domains

| | | |
|---|---|---|
| SEQ ID NO: 65 | humVx10_01 LC | DIVMTQSPLSLPVTPGEPASISCRSSQSLENSNGDTYLNWYLQKPGQSPRLLIYR |
| | variable | VSNRFSGVPDRFSGSGSGTDFTLKISRVEADDVGIYYCLQVSHVPWTFGQGTKLE |
| | | IK |
| SEQ ID NO: 66 | humVx14_01 LC | DIVMTQSPLSLPVTPGEPASISCRSSQSLVHSNGNTYLHWYLQKPGQSPRLLIYK |
| | variable | VSNRLSGVPDRFSGSGSGTDFTLKISRVEADDVGIYYCSQTTHVPYTFGQGTKLE |
| | | IK |
| SEQ ID NO: 67 | humVx14_02 LC | DVVMTQSPLSLPVTLGQPASISCRSSQSLVHSNGNTYLHWFQQRPGQSPRRLIYK |
| | variable | VSNRLSGVPDRFSGSGSGTDFTLKISRVEAEDVGVYYCSQTTHVPYTFGQGTKLE |
| | | IK |
| SEQ ID NO: 68 | humVx14_03 LC | DIVMTQSPDSLAVSLGERATINCRSSQSLVHSNGNTYLHWYQQKPGQPPKLLIYK |
| | variable | VSNRLSGVPDRFSGSGSGTDFTLTISSLQAEDVAVYYCSQTTHVPYTFGQGTKLE |
| | | IK |

Human Heavy Chain (HC) Variable Domains

| | | |
|---|---|---|
| SEQ ID NO: 69 | humVx10_01 HC | QVQLVQSGAEVKKPGASVQVSCKASGYTETNYWIHWLRQAPGQGLEWMGYIDPNT |
| | variable | VYTDYNQRFEDRVTMTSDTSISTAYMELSSLRSDDTAVYYCARGGKRGVDSWGQA |
| | | TLVTVSS |
| SEQ ID NO: 70 | humVx14_01 HC | QVQLVQSGAEVKKPGASVQVSCKASGYTFTNYGMNWLRQAPGQGLEWMGWININT |
| | variable | GEPTYAEDFKGRVTMTSDTSISTAYMELSSLRSDDTAVYYCARWARGGNFDLWGQ |
| | | ATLVTVSS |
| SEQ ID NO: 71 | humVx14_02 HC | EVQLVQSGAEVKKPGATVKISCKVSGYTFTNYGMNWVQQAPGKGLEWMGWININT |
| | variable | GEPTYAEDFKGRVTITADTSTDTAYMELSSLRSEDTAVYYCATWARGGNFDLWGQ |
| | | GTTVTVSS |
| SEQ ID NO: 72 | humVx14_03 HC | EVQLVQSGAEVKKPGESLKISCKGSGYTFTNYGMNWVRQMPGKGLEWMGWININT |
| | variable | GEPTYAEDFKGQVTISADKSISTAYLQWSSLKASDTAMYYCARWARGGNFDLWGQ |
| | | GTTVTVSS |
| SEQ ID NO: 73 | humVx14_04 HC | QVQLVQSGAEVKKPGASVKVSCKASGYTFTNYGMNWVRQAPGQGLEWMGWININT |
| | variable | GEPTYAEDFKGRVTMTTDTSTSTAYMELRSLRSDDTAVYYCARWARGGNFDLWGQ |
| | | GTTVTVSS |

Human Light Chains (LC)

| | | |
|---|---|---|
| SEQ ID NO: 74 | humVx10_LC01 | DIVMTQSPLSLPVTPGEPASISCRSSQSLENSNGDTYLNWYLQKPGQSPRLLIYR |
| | | VSNRFSGVPDRESGSGSGTDETLKISRVEADDVGIYYCLQVSHVPWTEGQGTKLE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 75 | humVx14_LC01 | DIVMTQSPLSLPVTPGHPASISCRSSQSLVHSNGNTYLHWYLQKPGQSPRLLIYK |
| | | VSNRLSGVPDRFSGSGSGTDFTLKISRVEADDVGIYYCSQTTHVPYTFGQGTKLE |
| | | IKRTVAAPSVFIFPPSDEQLKSGTASVVCLLNNFYPREAKVQWKVDNALWSGNSQ |
| | | ESVTEQDSKDSTYSLSSTLTLSKADYEKHKVYACEVTHQGLSSPVTKSFNRGEC |
| SEQ ID NO: 76 | humVx14_LC02 | DVVMTQSPLSLPVTLGQPASISCRSSQSLVHSNGNTYLHWFQQRPGQSPRRLIYK |
| | | VSNRLSGVPDRFSGSGSGTDFTLKISRVEAEDVGVYYCSQTTHVPYTFGQGTKLH |
| | | IKRTVAAPSVFIFPPSDHQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 77 | humVx14_LC03 | DIVMTQSPDSLAVSLGERATINCRSSQSLVHSNGNTYLHWYQQKPGQPPKLLIYK |
| | | VSNRLSGVPDRFSGSGSGTDFTLTISSLQAEDVAVYYCSQTTHVPYTFGQGTKLE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |

Human Heavy Chains (HC)

| | | |
|---|---|---|
| SEQ ID NO: 78 | humVx10_HC01 | QVQLVQSGAEVKKPGASVQVSCKASGYTFTNYWIHWLRQAPGQGLEWMGYIDPNT |
| | | VYTDYNQRFEDRVTMTSDTSISTAYMELSSLRSDDTAVYYCARGGKRGVDSWGQA |
| | | TLVTVSSASTKGPSVFPLAPCSRSTSESTAALGCLVKDYFPEPVTVSWNSGALTS |
| | | GVHTFPAVLQSSGLYSLSSVVTVPSSSLGTKTYTCNVDHKPSNTKVDKRVESKYG |
| | | PPCPPCPAPEFEGGPSVFLFPPKPKDTLMISRTPEVTCVVVDVSQEDPEVQFNWY |
| | | VDGVEVHNAKTKPREEQFNSTYRVVSVLTVLHQDWLNGKEYKCKVSNKGLPSSIE |
| | | KTISKAKGQPREPQVYTLPPSQEEMTKNQVSLTCLVKGFYPSDIAVEWESNGQPE |
| | | NNYKTTPPVLDSDGSFFLYSRLTVDKSRWQEGNVFSCSVMHEALHNHYTQKSLSL |
| | | SLG |
| SEQ ID NO: 79 | humVx14_HC01 | QVQLVQSGAEVKKPGASVQVSCKASGYTFTNYGMNWLRQAPGQGLEWMGWININT |
| | | GEPTYAEDFKGRVTMTSDTSISTAYMELSSLRSDDTAVYYCARWARGGNFDLWGQ |
| | | ATLVTVSSASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKVDKRVESKY |
| | | GPPCPPCPAPEFEGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNGQP |
| | | ENNYKTTPPVLDSDGSFFLYSRLTVDKSRWQEGNVFSCSVMHEALHNHYTQKSLS |
| | | LSLGASTKGPSVFPLAPCSRSTSESTAALGCLVKDYFPEPVTVSWNSGALTSGVH |
| | | TFPAVLQSSGLYSLSSVVTVPSSSLGTKTYTCNVDHKPSNTKVDKRVESKYGPPC |
| | | PPCPAPEFEGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSIEKTI |
| | | SKAKGQPREPQVYTLPPSQEEMTKNQVS |
| SEQ ID NO: 80 | humVx14_HC02 | EVQLVQSGAEVKKPGATVKISCKVSGYTFTNYGMNWVQQAPGKGLEWMGWININT |
| | | GEPTYAEDFKGRVTITADTSTDTAYMELSSLRSEDTAVYYCATWARGGNFDLWGQ |
| | | GTTVTVSSASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKVDKRVESKY |
| | | GPPCPPCPAPEFEGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNGQP |
| | | ENNYKTTPPVLDSDGSFFLYSRLTVDKSRWQEGNVFSCSVMHEALHNHYTQKSLS |
| | | LSLG |
| SEQ ID NO: 81 | humVx14_HC03 | EVQLVQSGAEVKKPGESLKISCKGSGYTFTNYGMNWVRQMPGKGLEWMGWININT |
| | | GEPTYAEDFKGQVTISADKSISTAYLQWSSLKASDTAMYYCARWARGGNFDLWGQ |
| | | GTTVTVSSASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKVDKRVESKY |
| | | GPPCPPCPAPEFEGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNGQP |
| | | ENNYKTTPPVLDSDGSFFLYSRLTVDKSRWQEGNVFSCSVMHEALHNHYTQKSLS |
| | | LSLG |
| SEQ ID NO: 82 | humVx14_HC04 | QVQLVQSGAEVKKPGASVKVSCKASGYTFTNYGMNWVRQAPGQGLEWMGWININT |
| | | GEPTYAEDFKGRVTMTTDTSTSTAYMELRSLRSDDTAVYYCARWARGGNFDLWGQ |
| | | GTTVTVSSASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKVDKRVESKY |
| | | GPPCPPCPAPEFEGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNGQP |
| | | ENNYKTTPPVLDSDGSFFLYSRLTVDKSRWQEGNVFSCSVMHEALHNHYTQKSLS |
| | | LSLG |

Human IgG-Fc
>Human Fc IgG1
ASTKGPSVFPLAPSSKSTSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEVKENWYVDGVEVHNAKT
KPREEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK
(SEQ ID NO: 83).
>Human Fc-IgG1-N297Q
ASTKGPSVFPLAPSSKSTSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK
(SEQ ID NO: 84).
>Human Fc-IgG2
ASTKGPSVFPLAPCSRSTSESTAALGCLVKDYFPEPVTVSWNSGALTSGVHTFPAVLQSSGLYSLSSVVTVPSSNEGTQTYTCNVD
HKPSNTKVDKTVERKCCVECPPCPAPPVAGPSVFLFPPKPKDTLMISRTPEVTCVVVDVSHEDPEVQFNWYVDGVEVHNAKTKPRE
EQFNSTFRVVSVLTVVHQDWLNGKEYKCKVSNKGLPAPIEKTISKTKGQPREPQVYTLPPSREEMTKNQVSLTCLVKGFYPSDIAV
EWESNGQPENNYKTTPPMLDSDGSFFLYSKLTVDKSRWQQGNVFSCSVMHEALHNHYTQKSLSLSPGK
(SEQ ID NO: 85).
>Human Fc-IgG3
ASTKGPSVFPLAPCSRSTSGGTAALGCLVKDYFPEPVTVSWNSGALTSGVHTFPAVLQSSGLYSLSSVVTVPSSSLGTQTYTCNVN
HKPSNTKVDKRVELKTPLGDTTHTCPRCPEPKSCDTPPPCPRCPEPKSCDTPPPCPRCPEPKSCDTPPPCPRCPAPELLGGPSVFL
FPPKPKDTLMISRTPEVTCVVVDVSHEDPEVQFKWYVDGVEVHNAKTKPREEQYNSTFRVVSVLTVLHQDWLNGKEYKCKVSNKAL
PAPIEKTISKTKGQPREPQVYTLPPSREEMTKNQVSLTCLVKGFYPSDIAVEWESSGQPENNYNTTPPMLDSDGSFFLYSKLTVDK
SRWQQGNIFSCSVMHELAHNRFTQKSLSLSPGK (SEQ ID NO: 86)
>Human Fc-IgG4
ASTKGPSVFPLAPCSRSTSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLG (SEQ ID NO: 87).
>Human Fc-IgF4 S228P
ASTKGPSVFPLAPCSRSTSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLG (SEQ ID NO: 88).
>Human Fc-IgG4 PE
ASTKGPSVFPLAPCSRSTSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFEGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK (SEQ ID NO: 89)
>Human Fc-IgG4 PE′
ASTKGPSVFPLAPCSRSTSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFEGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLG (SEQ ID NO: 90)
>Human kappa LC
RTVAAPSVFIFPPSDEQLKSGTASVVCLLNNFYPREAKVQWKVDNALQSGNSQESVTEQDSKDSTYSLSSTLTLSKADYEKHKVYA
CEVTHQGLSSPVTKSFNRGEC (SEQ ID NO: 91).
>Rat Fc-IgG2c
ARTTAPSVYPLVPGCSGTSGSLVTLGCLVKGYFPEPVTVKWNSGALSSGVHTFPAVLQSGLYTLSSSVTVPSSTWSSQTVTCSVAH
PATKSNLIKRIEPRRPKPRPPTDICSCDDNLGRPSVFIFPPKPKDILMITLTPKVTCVVVDVSEEEPDVQFSWFVDNVRVFTAQTQ
PHEEQLNGTFRVVSTLHIQHQDWMSGKEFKCKVNNKDLPSPIEKTISKPRGKARTPQVYTIPPPREQMSKNKVSLTCMVTSFYPAS
ISVEWERNGELEQDYKNTLPVLDSDESYFLYSKLSVDTDSWMRGDIYTCSVVHEALHNHHTQKNLSRSPGK
(SEQ ID NO: 92).
>Rat kappa LC
RADAAPTVSIFPPSMEQLTSGGATVVCFVNNFYPRDISVKWKIDGSEQRDGVLDSVTDQDSKDSTYSMSSTLSLTKVEYERHNLYT
CEVVHKTSSSPVVKSFNRNEC (SEQ ID NO: 93).
>Rabbit IgG-Fc
GQPKAPSVFPLAPCCGDTPSSTVTLGCLVKGYLPEPVTVTWNSGTLTNGVRTFPSVRQSSGLYSLSSWSVTSSSQPVTCNVAHPAT
NTKVDKTVAPSTCSKPTCPPPELLGGPSVFIFPPKPKDTLMISRTPEVTCVVVDVSQDDPEVQFTWYINNEQVRTARPPLREQQFN
STIRVVSTLPIAHQDWLRGKEFKCKVHNKALPAPIEKTISKARGQPLEPKVYTMGPPREELSSRSVSLTCMINGFYPSDISVEWEK
NGKAEDNYKTTPAVLDSDGSYFLYSKLSVPTSEWQRGDVFTCSVMHEALHNHYTQKSISRSPGK (SEQ ID NO: 94).
>Rabbit kappa LC
RDPVAPTVLIFPPAADQVATGTVTIVCVANKYFPDVTVTWEVDGTTQITGIHNSKTPQNSADCTYNLSSTLTLTSTQYNSHKHYTC
KVTQGTTSVVQSFNRGDC (SEQ ID NO: 95).
>CD47
MWPLVAALLLGSACCGSAQLLFNKTKSVEFTFCNDTVVIPCFVTNMEAQNTTEVYVKWKFKGRDIYTFDGALNKSTVPTDFSSAKI
EVSQLLKGDASLKMDKSDAVSHTGNYTCEVTELTREGETIIELKYRVVSWFSPNENILIVIFPIFAILLFWGQFGIKTLKYRSGGM
DEKTIALLVAGLVITVIVIVGAILFVPGEYSLKNATGLGLIVTSTGILILLHYYVFSTIAGLTSFVIALILVIQVIAYILAVVGLS
LCIAACIPMHGPLLISGLSILALAQLLGLVYMKFVE (SEQ ID NO: 96).

### Example 2

### Production of CD47 Antibodies

Chimeric antibodies disclosed herein comprise a mouse heavy chain variable domain and a light chain variable domain combined with a human kappa or human Fc IgG1, IgG1-N297Q, IgG2, IgG4, IgG4 S228P, IgG4 PE, and IgG4 PE* constant domains, respectively. These were designed to incorporate a secretion signal, cloned into a mammalian expression system and transfected into CHO cells to generate chimeric antibodies. The chimeric variants were expressed as full length IgG molecules, secreted into the medium, and purified using protein A.

As such, the humanized antibodies disclosed herein comprise frameworks derived from the human genome. The collection covers the diversity found in the human germ line sequences, yielding functionally expressed antibodies in vivo. The complementarity determining regions (CDRs) in the light and heavy chain variable regions of the murine and chimeric antibodies are described herein and were determined by following commonly accepted rules disclosed in “Protein Sequence and Structure Analysis of Antibody Variable Domains”, In: Antibody Engineering Lab Manual, eds. S. Duebel and R. Kontermann, Springer-Verlag, Heidelberg (2001)). The human light chain variable domains were then designed. The humanized variable domains were then combined with a secretion signal and human kappa and human Fc IgG1, IgG1-N297Q, IgG2, IgG3, IgG4 S228P, IgG4 PE, and IgG4 PE′ constant domains, cloned into a mammalian expression system, and transfected into CHO cells to generate humanized mAbs. The humanized variants were expressed as full length IgG molecules, secreted into the medium and purified using protein A.

A non-glycosylated version (IgG1-N297Q) was created by site directed mutagenesis of heavy chain position 297 to change the asparagine to glutamine (Human Fc IgG1-N297Q, SEQ ID NO:84). An IgG4 variant was created by site-directed mutagenesis at position 228 to change the serine to proline thereby preventing in vivo Fab arm exchange. An IgG4 double mutant was created by site-directed mutagenesis at positions 228 (serine to proline) and 235 (leucine to glutamate) to prevent Fab arm exchange and to further reduce Fc effector function. IgG2, IgG3, IgG4 S228P, and IgG4 PE isotypes were constructed by cloning the heavy chain variable domain in frame with the human IgG2, IgG3, IgG4 S228P, and IgG4PE constant domains (Human Fc-IgG2, SEQ ID NO:85; Human Fc-IgG3, SEQ ID NO:86; Human Fc-IgG4 S228P, SEQ ID NO:88; and Human Fc-IgG4 PE, SEQ ID NO:89); Human Fc-IgG4 PE′; SEQ ID NO:90.

### Example 3

### Binding of CD47 Monoclonal Antibodies (mAbs)

The binding of murine, chimeric, and humanized antibodies of the present disclosure was determined by flow cytometry using freshly isolated red blood cells from mouse, human, pig, dog, or rat RBCs, which display CD47 on their surface (Kamel et al. 2010. Blood Transfus. 8(4):260-266) or by ELISA using OVIO cells transfected with human CD47 (OVIO-hCD47).

Binding activities of murine mAbs to mouse CD47 on murine RBCs (mRBCs) and human CD47 on human RBCs (hRBCs) were determined using flow cytometry. RBCs were incubated for 60 min on at 37° C. with various concentrations of the chimeric or humanized antibodies in a solution of phosphate buffered saline, pH 7.2, 2.5 mM EDT A (PBS+E). Cells were then washed with cold PBS+E and incubated for an additional hour on ice with a FITC labeled goat-anti-mouse antibody (Jackson Immuno Research Labs, West Grove, Pa.) in PBS +E. Cells were washed with PBS+E, antibody binding analyzed using a C6 Accuri How Cytometer (Becton Dickinson) and apparent binding affinities determined by non-linear fit (Prism GraphPad software) of the median fluorescence intensities at the various antibody concentrations.

Binding activities of humanized mAbs to human CD47 on human RBCs were determined using flow cytometry. RBCs were incubated for 60 min on at 37° C. with various concentrations of the chimeric or humanized antibodies in a solution of phosphate buffered saline, pH 7.2, 2.5 mM EDT A (PBS+E). Cells were then washed with cold PBS+E and incubated for an additional hour on ice with a FITC labeled donkey-anti-human antibody (Jackson Immuno Research Labs, West Grove, Pa.) in PBS+E. Cells were washed with PBS+E, antibody binding analyzed using a C6 Accuri Flow Cytometer (Becton Dickinson) and apparent binding affinities determined by non-linear fit (Prism GraphPad software) of the median fluorescence intensities at the various antibody concentrations.

Binding activities of humanized mAbs were determined using a cell-based ELISA assay with human OVIO-hCD47 cells expressing cell surface human CD47. OVIO hCD47 cells were grown in IMDM medium containing 10% heat inactivated fetal bovine serum (BioWest; S01520). One day before assay, 3×10⁴ cells were plated in 96 well cell bind plates (Corning #3300, VWR #66025-626) and were 95-100% confluent at the time of assay. Cells were washed, and various concentrations of purified antibodies added in IMDM 37° C. for 1 hr in 95%02 I 5% CO₂. Cells were then washed with media and incubated for an additional hour at 37° C. with HRP labeled secondary anti-human antibody (Promega) diluted 1/2500 in media. Cells were washed three times with PBS, and the peroxidase substrate 3,3′, 5,5′-tetramethylbenzidine is added (Sigma; Catalog #T4444). Reactions were terminated by the addition of HCl to 0.7N, and absorbance at 450 nM is determined using a Tecan model Infinite M200 plate reader. The apparent binding affinities of these clones to human OVIO-hCD47 cells was determined by non-linear fit (Prism GraphPad software).

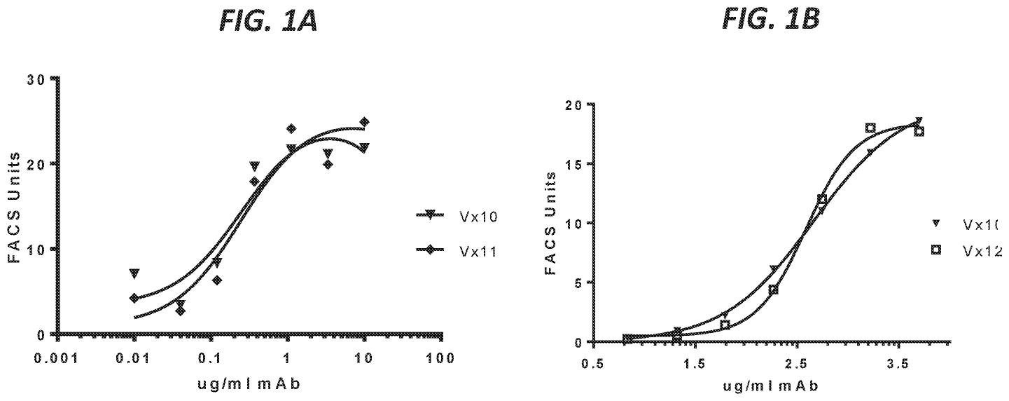

All of the murine mAbs bound to human hCD47 on hRBCs with apparent affinities in the picomolar (pM) range (FIG. 1B and Table 1). All of the murine mAbs showed cross-species binding with varying affinities observed for RBCs obtained from other species (FIG. 1A and Table 1).

TABLE 1
Anti-CD47 Mouse mAbs Bind to CD47 from Multiple Species.

| mAb | Human | Pig | Dog | Rat | Mouse |
|---|---|---|---|---|---|
| Vx10 | 459 | 123 | 714 | 179 | 31 |
| Vx11 | 91 | >10,000 | <13 | 231 | 246 |
| Vx12 | 384 | 60 | 101 | >10,000 | <13 |
| Vx13 | 874 | 102 | 1620 | >10,000 | >10,000 |

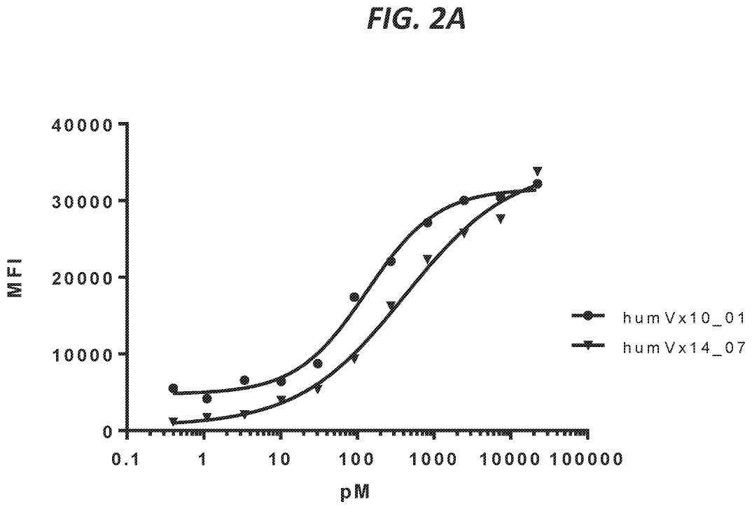

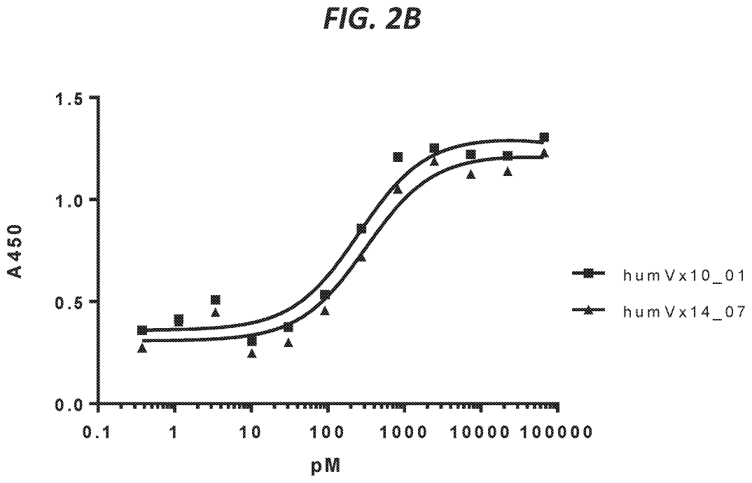

Similarly, the chimeric and humanized mAbs bound to hRBCs and to human OVI0 hCD47 tumor cells in a concentration-dependent manner (Table 2, FIG. 2A and FIG. 2B) with apparent affinities in the picomolar nanomolar range.

TABLE 2
CD47 Apparent Binding Affinity for Human-
Mouse Dual-Function Chimeric mAbs.

| | Kd (pM) | |
|---|---|---|
| | Human Tumor | Kd (pM) |
| | Cell-based | Human |
| | ELISA | RBC |
| Vx10_mh_IgG1N297Q | 87 | 40 |
| Vx10_mh_IgG4PE | 91 | 48 |
| Vx12_mh_IgG1N297Q | 69 | 90 |
| Vx12_mh_IgG4PE | 70 | 120 |

### Example 4

### CD47 mAbs Reverse TSP-1 Inhibition of NO-Stimulated cGMP Production in Jurkat Cells

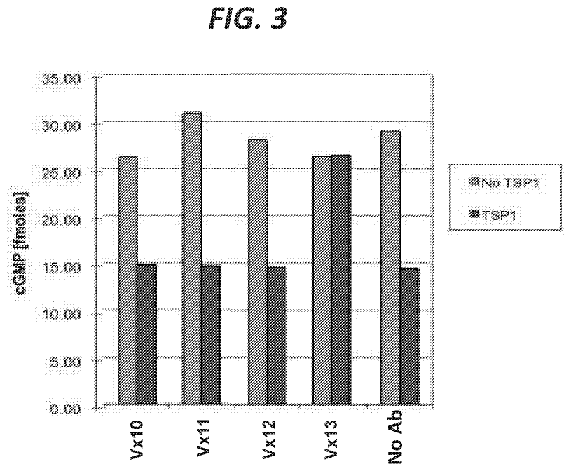

TSP1 is a potent inhibitor of NO-stimulated cGMP production (Isenberg, PNAS Sep. 13, 2005. 102 (37) 13141-13146) and may inhibit angiogenic responses at the level of this second messenger. DEA/NO transiently induces cGMP levels in Jurkat cells however, addition of 100 pM TSP1 inhibits the NO-stimulated increase in cGMP. Jurkat JE6.1 cells were incubated overnight in serum-free medium followed by incubation with 10 ug/ml Vx10, Vx11, Vx12, Vx13, or no antibody with or without TSP1. DEA/NO was subsequently added and cGMP levels were detected by ELISA (Cayman Chemical). Vx13 potently reversed TSP inhibition of cGMP production, whereas Vx10, Vx11, Vx12 and no antibody treatment did not (FIG. 3).

### Example 5

### CD47 Antibodies Block CD47/SIRPα Binding

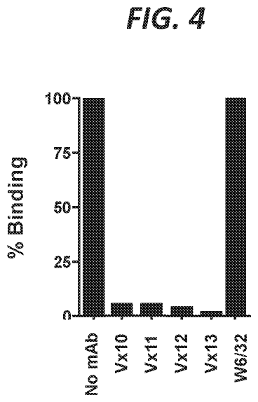

To assess the effect of mouse CD47 mAbs on binding of CD47 to SIRPα in vitro the following method is employed using the binding of fluorescently-labelled SIRPα-Fc fusion protein to CD47 expressing Jurkat T cells. SIRPα-Fc fusion protein (R&D Systems, cat #4546-SA) was labelled using an Alexa Fluor® antibody labelling kit (Invitrogen Cat No. A20186) according to the manufacturers specifications. 1.5×10⁶ Jurkat T cells were incubated with CD47 mAbs (5 μg/ml) or a control antibody in RPMI containing 10% media or media alone for 30 min at 37° C. An equal volume of fluorescently labeled SIRPα-Fc fusion protein was added and incubated for an additional 30 min at 37° C. Cells were washed once with PBS and the amount of labelled SIRPα-Fc bound to the Jurkat T cells analyzed by flow cytometry. As shown in FIG. 4, the all of the mouse CD47 mAbs, blocked the interaction of CD47 expressed on the Jurkat T cells with SIPRα, while the control antibody W6/32 (which does not bind to CD47) or media alone, did not block the CD47/SIRPα interaction.

### Example 6

### CD47 Antibodies Increase Phagocytosis

To assess the effect of mouse, chimeric and humanized CD47 mAbs on phagocytosis of tumor cells by macrophages in vitro the following method is employed using flow cytometry (Willingham et al. (2012) Proc Natl Acad Sci USA 109(17):6662-7 and Tseng et al. (2013) Proc Natl Acad Sci USA 110(27): 11103-8).

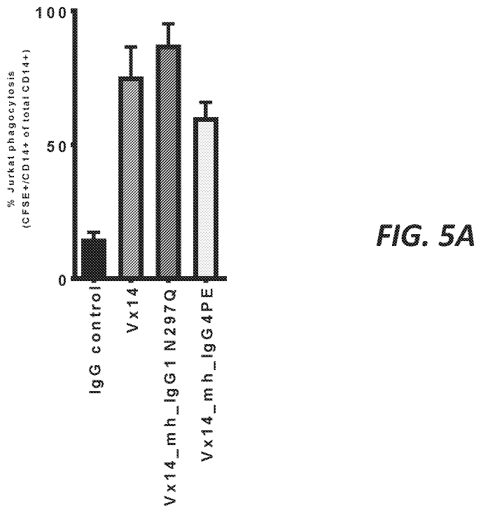

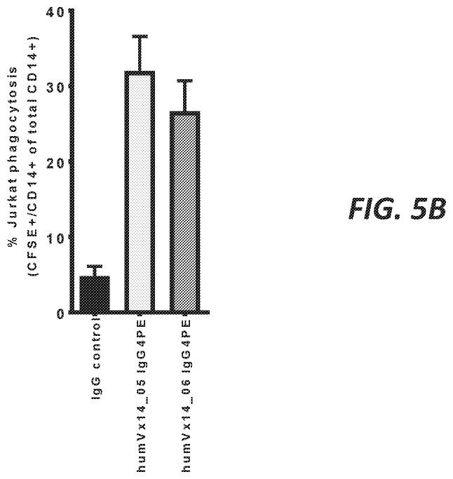

Human derived macrophages were derived from leukapheresis of healthy human peripheral blood and incubated in AIM-V media (Life Technologies) for 7-10 days. For the in vitro phagocytosis assay, macrophages were re-plated at a concentration of 1×10⁴ cells per well in 100 μl of AIM-V media in a 96-well plate and allowed to adhere for 24 hrs. Once the effector macrophages adhered to the culture dish, the target human cancer cells (Jurkat) were labeled with 1 μM 5(6)-Carboxyfluorescein diacetate N-succinimidyl ester (CFSE; Sigma Aldrich) and added to the macrophage cultures at a concentration of 5×10⁴ cells in 1 ml of AIM-V media (5: 1 target to effector ratio). CD47 mAbs (1 μg/ml) were added immediately upon mixture of target and effector cells and allowed to incubate at 37° C. for 2-3 hours. After 2-3 hrs, all non-phagocytosed cells were removed, and the remaining cells washed three times with phosphate buffered saline (PBS; Sigma Aldrich). Cells were then trypsinized, collected into microcentrifuge tubes, and incubated in 100 ng of allophycocyanin (APC) labeled CD14 antibodies (BD Biosciences) for 30 minutes, washed once, and analyzed by flow cytometry (Accuri C6; BD Biosciences) for the percentage of CD14⁺ cells that were also CFSE⁺ indicating complete phagocytosis. As shown in FIG. 5A, the mouse Vx14 and chimeric Vx14_mh_IgG1N297Q and Vx14_mh_IgG4PE CD47 mAbs increased phagocytosis of Jurkat cells by human macrophages by blocking the CD47/SIRPα interaction and this enhanced phagocytosis is independent of Fc function. Similarly, as shown in FIG. 5B, humanized CD47 mAbs humVx14_05 IgG4PE and humVx14_06 IgG4PE) and increased phagocytosis of Jurkat cells by human macrophages by blocking the CD47/SIRPα interaction.

### Example 7

### Induction of Cell Death by Soluble CD47 Antibodies

Some soluble CD47 antibodies have been shown to induce selective cell death of tumor cells. This additional property of selective toxicity to cancer cells is expected to have advantages compared to mAbs that only block SIRPα binding to CD47.

Induction of cell death by soluble anti-CD47 mAbs is measured in vitro (Manna et al. (2003) J Immunol. 107 (7): 3544-53). For the in vitro cell death assay, 1×10⁵ transformed human T cells (Jurkat T cells) were incubated with soluble chimeric Vx1027xi and humanized hum1002C and hum 1027C for 24 hrs at 37° C. As cell death occurs, mitochondrial membrane potential is decreased, the inner leaflet of the cell membrane is inverted exposing phosphatidylserines (PS), and propidium iodide (PI) is able to incorporate into nuclear DNA. In order to detect these cellular changes, cells were stained with fluorescently labeled annexin V and PI or 7-aminoactinomycin D (7-AAD) (BD Biosciences) and the signal was detected using an Accuri C6 flow cytometer (BD Biosciences). The increase in PS exposure is determined by measuring the percent increase in annexin V signal and the percent of dead cells by measuring the percent increase in PI or 7-AAD signal. These mAbs induce cell death of tumor cells directly and do not require complement or the intervention of other cells (e.g., NK cells, T cells, or macrophages) to kill Thus, the mechanism is independent of both other cells and of Fe effector function. Therefore, therapeutic antibodies developed from these mAbs can be engineered to reduce Fe effector functions such as ADCC and CDC and thereby limit the potential for side effects common to humanized mAbs with intact Fc effector functions.

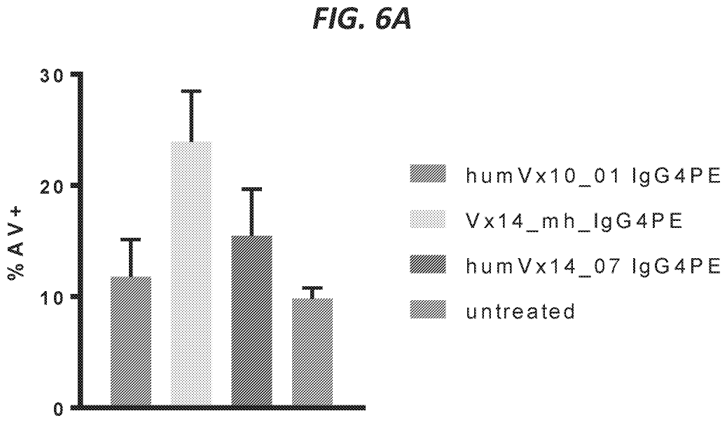

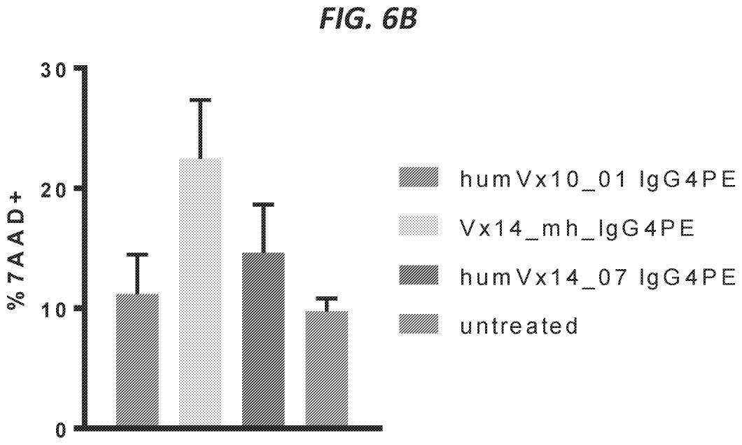

As shown in FIG. 6A and FIG. 6B, the chimeric Vx14_mh_IgG4PE and the soluble humanized CD47 mAbs (humVx10_01 IgG4PE and humVx14_07 IgG4PE) induced cell death of Jurkat T ALL cells as measured by increased annexin V staining and 7-AAD staining. Induction of cell death and the promotion of phagocytosis of susceptible cancer cells imparts an additional desirable antibody property and therapeutic benefit in the treatment of cancer.

TABLE 3

| | SEQ ID NOs: | | | |
|---|---|---|---|---|
| | | Heavy | Light | Heavy |
| | Light Chain | Chain | Chain | Chain |
| | Variable | Variable | Full | Full |
| Antibody | Domain | Domain | Length | Length |
| Vx10 | 39 | 48 | | |
| Vx11 | 40 | 49 | | |
| Vx12 | 41 | 50 | | |
| Vx13 | 42 | 51 | | |
| Vx14 | 38 | 47 | | |
| Vx10_mh_IgG1N297Q | 39 | 48 | 56 | 57 |
| Vx10_mh_IgG4PE | 39 | 48 | 56 | 58 |
| Vx12_mh_IgG1N297Q | 41 | 50 | 59 | 60 |
| Vx12_mh_IgG4PE | 41 | 50 | 59 | 61 |
| Vx14_mh_IgG1N297Q | 38 | 47 | 62 | 63 |
| Vx14_mh_IgG4PE | 38 | 47 | 62 | 64 |
| humVx14_07_IgG4PE | 66 | 70 | 75 | 79 |
| humVx10_01_IgG4PE | 65 | 69 | 74 | 78 |
| humVx14_05_IgG4PE | 68 | 72 | 77 | 81 |
| humVx14_06_IgG4PE | 68 | 73 | 77 | 82 |

1. A monoclonal antibody or antigen binding fragment thereof, of claim 1, wherein the monoclonal antibody, or antigen binding fragment comprises one or more of the characteristics:
i. blocks the interaction between CD47 and its ligand SIRPα
ii. increases phagocytosis of human tumor cells
iii. induces death of susceptible human tumor cells; and
iv. reverses TSP1 inhibition of the nitric oxide (NO) pathway.

2. The monoclonal antibody or antigen-binding fragment thereof, of claim 1, that specifically binds human, rat, mouse, pig and/or cynomolgus monkey CD47.

3. The monoclonal antibody, or antigen-binding fragment thereof, of claims 1-2, comprising three light chain complementarity determining regions (LCDR1, LCDR2, LCDR3) and three heavy chain complementarity determining regions (HCDR1, HCDR2, HCDR3), wherein the three light chain complementarity determining regions (LCDR1, LCDR2, LCDR3) are selected from:

| LCDR1 | | LCDR2 | | LCDR3 | |
|---|---|---|---|---|---|
| SEQ ID NO: 1 | RSSQSLVHSNGNTYLH | SEQ ID NO: 7 | KVSNRLS | SEQ ID NO: 11 | SQTTHVPYT |
| SEQ ID NO: 2 | RSSQSLENSNGDTYLN | SEQ ID NO: 8 | RVSNRFS | SEQ ID NO: 12 | LQVSHVPWT |
| SEQ ID NO: 1 | RSSQSLVHSNGNTYLH | SEQ ID NO: 9 | KVSNRFS | SEQ ID NO: 13 | SQSTHVPRT |
| SEQ ID NO: 1 | RSSQSLVHSNGNTYLH | SEQ ID NO: 10 | KVSNRFS | SEQ ID NO: 14 | SQSTHVLT |
| SEQ ID NO: 4 | RSSQNIVQSNGNTYLE | SEQ ID NO: 9 | KVFHRFS | SEQ ID NO: 15 | FQGSHVPWT |
| SEQ ID NO: 4 | RSSQNIVQSNGNTYLE | SEQ ID NO: 9 | KVFHRFS | SEQ ID NO: 16 | FQGSYVPTW |
| SEQ ID NO: 5 | RASSSIFYVD | SEQ ID NO: 10 | DTSKLAS | SEQ ID NO: 17 | QQSWWNPPT |
| SEQ ID NO: 6 | SASSSIFYVD | SEQ ID NO: 10 | DTSKLAS | SEQ ID NO: 17 | QQWSSNPPT |

and the three heavy chain complementarity determining regions (HCDR1, HCDR2, HCDR3) are selection from:

| HCDR1 | | HCDR2 | | HCDR3 | |
|---|---|---|---|---|---|
| SEQ ID NO: 18 | GYTFTNYGMN | SEQ ID NO: 24 | WININTGEPTYADEFKG | SEQ ID NO: 31 | WARGGNFDL |
| SEQ ID NO: 19 | GYTFTNYWIH | SEQ ID NO: 25 | YIDPNTVYTDYNQRFED | SEQ ID NO: 32 | GGKRGVDS |
| SEQ ID NO: 20 | GYTFTNYFLH | SEQ ID NO: 26 | DINPNAGSTNLNERFKS | SEQ ID NO: 33 | GGTMDY |
| SEQ ID NO: 20 | GYTFTNYFLY | SEQ ID NO: 26 | DINPNAGSTNLNERFKS | SEQ ID NO: 34 | GGYTMDY |
| SEQ ID NO: 21 | DYTFTNYYIH | SEQ ID NO: 27 | WIYPGNNNNKYNEKFKG | SEQ ID NO: 34 | GGYTMDY |
| SEQ ID NO: 22 | GYFTFNYWMH | SEQ ID NO: 28 | YIDPRTAYTEYNQKFKD | SEQ ID NO: 35 | GGRVGLGY |
| SEQ ID NO: 22 | GYTFTNYWMH | SEQ ID NO: 29 | YIDPRTDYSEYNQKFKD | SEQ ID NO: 35 | GGRVGLGY |
| SEQ ID NO: 23 | GYSFTGYYMH | SEQ ID NO: 30 | RANPYNGGTSYNQKFKG | SEQ ID NO: 36 | NYGGSDAMDY |
| SEQ ID NO: 23 | GYSFTGYYMH | SEQ ID NO: 30 | RANPYNGGTSYNQKFKG | SEQ ID NO: 37 | NYGSSDAMDY |

4. The monoclonal antibody or antigen binding fragment thereof, of claims 1-3, comprising a combination of a heavy chain variable domain (V_{H}) and light chain variable domain (V_{L}), wherein the combination is selected from the group consisting of:
i. a heavy chain variable domain comprising the amino acid sequence of SEQ ID NO:47 and a light chain variable domain comprising the amino acid sequence SEQ ID NO:38;
ii. a heavy chain variable domain comprising the amino acid sequence of SEQ ID NO:48 and a light chain variable domain comprising the amino acid sequence SEQ ID NO:39;
iii. a heavy chain variable domain comprising the amino acid sequence of SEQ ID NO:49 and a light chain variable domain comprising the amino acid sequence SEQ ID NO:40;
iv. a heavy chain variable domain comprising the amino acid sequence of SEQ ID NO:50 and a light chain variable domain comprising the amino acid sequence SEQ ID NO:41;
v. a heavy chain variable domain comprising the amino acid sequence of SEQ ID NO:51 and a light chain variable domain comprising the amino acid sequence SEQ ID NO:42;
vi. a heavy chain variable domain comprising the amino acid sequence of SEQ ID NO:52 and a light chain variable domain comprising the amino acid sequence SEQ ID NO:43;
vii. a heavy chain variable domain comprising the amino acid sequence of SEQ ID NO:53 and a light chain variable domain comprising the amino acid sequence SEQ ID NO:44;
viii. a heavy chain variable domain comprising the amino acid sequence of SEQ ID NO:54 and a light chain variable domain comprising the amino acid sequence SEQ ID NO:45;
ix. a heavy chain variable domain comprising the amino acid sequence of SEQ ID NO:55 and a light chain variable domain comprising the amino acid sequence SEQ ID NO:46;
x. a heavy chain variable domain comprising the amino acid sequence of SEQ ID NO:70 and a light chain variable domain comprising the amino acid sequence SEQ ID NO:66;
xi. a heavy chain variable domain comprising the amino acid sequence of SEQ ID NO:69 and a light chain variable domain comprising the amino acid sequence SEQ ID NO:65;
xii. a heavy chain variable domain comprising the amino acid sequence of SEQ ID NO:72 and a light chain variable domain comprising the amino acid sequence SEQ ID NO:68;
xiii. a heavy chain variable domain comprising the amino acid sequence of SEQ ID NO:73 and a light chain variable domain comprising the amino acid sequence SEQ ID NO:68;
xiv. a heavy chain variable domain comprising the amino acid sequence of SEQ ID NO:71 and a light chain variable domain comprising the amino acid sequence SEQ ID NO:67;
xv. a heavy chain variable domain comprising the amino acid sequence of SEQ ID NO:72 and a light chain variable domain comprising the amino acid sequence SEQ ID NO:67;
xvi. a heavy chain variable domain comprising the amino acid sequence of SEQ ID NO:73 and a light chain variable domain comprising the amino acid sequence SEQ ID NO:67; and
xvii. a heavy chain variable domain comprising the amino acid sequence of SEQ ID NO:71 and a light chain variable domain comprising the amino acid sequence SEQ ID NO:68.

5. The monoclonal antibody or antigen binding fragment thereof, of claims 1-4, comprising at least one heavy chain and at least one light chain selected from the selected from the group consisting of:
i. a heavy chain comprising the amino acid sequence of SEQ ID NO:57 and a light chain comprising the amino acid sequence SEQ ID NO:56;
ii. a heavy chain comprising the amino acid sequence of SEQ ID NO:58 and a light chain comprising the amino acid sequence SEQ ID NO:56;
iii. a heavy chain comprising the amino acid sequence of SEQ ID NO:60 and a light chain comprising the amino acid sequence SEQ ID NO:59;
iv. a heavy chain comprising the amino acid sequence of SEQ ID NO:61 and a light chain comprising the amino acid sequence SEQ ID NO:59;
v. a heavy chain comprising the amino acid sequence of SEQ ID NO:63 and a light chain comprising the amino acid sequence SEQ ID NO:62;
vi. a heavy chain comprising the amino acid sequence of SEQ ID NO:64 and a light chain comprising the amino acid sequence SEQ ID NO:62;
vii. a heavy chain comprising the amino acid sequence of SEQ ID NO:79 and a light chain comprising the amino acid sequence SEQ ID NO:75;
viii. a heavy chain comprising the amino acid sequence of SEQ ID NO:78- and a light chain comprising the amino acid sequence SEQ ID NO:74;
ix. a heavy chain comprising the amino acid sequence of SEQ ID NO:81 and a light chain comprising the amino acid sequence SEQ ID NO:77;
x. a heavy chain comprising the amino acid sequence of SEQ ID NO:82 and a light chain comprising the amino acid sequence SEQ ID NO:77;
xi. a heavy chain comprising the amino acid sequence of SEQ ID NO:80 and a light chain comprising the amino acid sequence SEQ ID NO:76;
xii. a heavy chain comprising the amino acid sequence of SEQ ID NO:81 and a light chain comprising the amino acid sequence SEQ ID NO:76;
xiii. a heavy chain comprising the amino acid sequence of SEQ ID NO:82 and a light chain comprising the amino acid sequence SEQ ID NO:76; and
xiv. a heavy chain comprising the amino acid sequence of SEQ ID NO:80 and light chain comprising the amino acid sequence SEQ ID NO:77.

6. An antibody or antigen binding fragment thereof, of any of the of the preceding claims, wherein the antibody or antigen binding fragment thereof is a murine, chimeric, or humanized antibody.

7. The monoclonal antibody or antigen binding fragment thereof, of claims 1-6, wherein the monoclonal antibody, or antigen binding fragment thereof causes complete reversal of NO pathway inhibition.

8. The antibody or antigen binding fragment thereof, of claims 1-6, wherein the monoclonal antibody, or antigen binding fragment thereof causes intermediate reversal of NO pathway inhibition.

9. The antibody, or antigen binding fragment thereof of claims 1-6, wherein the monoclonal antibody, or antigen binding fragment thereof causes no reversal of NO pathway inhibition.

10. The monoclonal antibody or antigen binding fragment thereof, of any one of claims 1-9, which displays one or more effector functions selected from antibody-dependent cellular cytotoxicity (ADCC), complement-dependent cytotoxicity (CDC), antibody-dependent cellular phagocytosis (ADCP), and C1q binding against CD47-expressing cancer cells.

11. A pharmaceutical composition, comprising said monoclonal antibody or antigen binding fragment thereof, of any one of claims 1-10, and a pharmaceutically or physiologically acceptable carrier, diluent, or excipient.

12. The monoclonal antibody or antigen binding fragment thereof, of claim 11, for use in human therapy.

13. The monoclonal antibody or antigen binding fragment thereof, for use according to claim 12, for use in reducing, preventing, and/or treating ischemia-reperfusion injury, or an autoimmune, autoinflammatory, inflammatory or cardiovascular disease.

14. The monoclonal antibody or antigen binding fragment thereof, for use according to claim 12, wherein the subjects to be treated are a human, a companion/pet animal, working animal, sport animal, zoo animal, or other valuable animal kept in captivity.

15. The monoclonal antibody, or antigen binding fragment thereof, for use according claim 13, wherein said ischemia-reperfusion injury occurs in organ transplantation, acute kidney injury, cardiovascular disease, cardiopulmonary bypass surgery, pulmonary hypertension, sickle cell disease, coronary heart disease, coronary artery disease, myocardial infarction, cerebrovascular disease, stroke, surgical resections and reconstructive surgery, reattachment of an appendage or other body part, skin grafting, or trauma.

16. The monoclonal antibody or antigen binding fragment thereof, for use according to claim 12, for use, in reducing, preventing, and/or treating heart failure.

17. The monoclonal antibody, or antigen binding fragment thereof, for use according to claim 13, wherein said autoimmune, autoinflammatory, or inflammatory disease is selected from the group consisting of arthritis, rheumatoid arthritis, multiple sclerosis, psoriasis, psoriatic arthritis, Crohn's disease, inflammatory bowel disease, ulcerative colitis, lupus, systemic lupus erythematous, juvenile rheumatoid arthritis, juvenile idiopathic arthritis, Grave's disease, Hashimoto's thyroiditis, Addison's disease, celiac disease, dermatomyositis, multiple sclerosis, myasthenia gravis, pernicious anemia, Sjogren syndrome, type I diabetes, vasculitis, uveitis, atherosclerosis and ankylosing spondylitis.

18. The monoclonal antibody or antigen binding fragment thereof, for use according to claim 12, in preventing or treating cancer in a human patient.

19. The monoclonal antibody or antigen binding fragment thereof, of claim 12, which increases phagocytosis of tumor cells of said cancer.

20. The monoclonal antibody or antigen binding fragment thereof, of claim 18, wherein said cancer is selected from the group consisting of a leukemia, a lymphoma, multiple myeloma, ovarian cancer, breast cancer, endometrial cancer, colon cancer (colorectal cancer), rectal cancer, bladder cancer, urothelial cancer, lung cancer (non-small cell lung cancer, adenocarcinoma of the lung, squamous cell carcinoma of the lung), bronchial cancer, bone cancer, prostate cancer, pancreatic cancer, gastric cancer, hepatocellular carcinoma, gall bladder cancer, bile duct cancer, esophageal cancer, renal cell carcinoma, thyroid cancer, squamous cell carcinoma of the head and neck (head and neck cancer), testicular cancer, cancer of the endocrine gland, cancer of the adrenal gland, cancer of the pituitary gland, cancer of the skin, cancer of soft tissues, cancer of blood vessels, cancer of brain, cancer of nerves, cancer of eyes, cancer of meninges, cancer of oropharynx, cancer of hypopharynx, cancer of cervix, and cancer of uterus, glioblastoma, meduloblastoma, astrocytoma, glioma, meningioma, gastrinoma, neuroblastoma, melanoma, myelodysplastic syndrome, and a sarcoma.

21. The monoclonal antibody or antigen binding fragment thereof, of claim 20, wherein said leukemia is selected from the group consisting of systemic mastocytosis, acute lymphocytic (lymphoblastic) leukemia (ALL), T cell-ALL, acute myeloid leukemia (AML), myelogenous leukemia, chronic lymphocytic leukemia (CLL), chronic myeloid leukemia (CML), myeloproliferative disorder/neoplasm, myelodysplastic syndrome, monocytic cell leukemia, and plasma cell leukemia; wherein said lymphoma is selected from the group consisting of histiocytic lymphoma and T cell lymphoma, B cell lymphomas, including Hodgkin's lymphoma and non-Hodgkin's lymphoma, such as low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, and Waldenstrom's Macroglobulinemia; and wherein said sarcoma is selected from the group consisting of osteosarcoma, Ewing's sarcoma, leiomyosarcoma, synovial sarcoma, alveolar soft part sarcoma, angiosarcoma, liposarcoma, fibrosarcoma, rhabdomyosarcoma, and chrondrosarcoma.

22. A monoclonal antibody, or antigen binding fragment thereof, for use in a method of treating cancer, wherein said monoclonal antibody or antigen binding fragment, binds CD47 on a human tumor cell and thereby prevents the binding of said CD47 to a SIRPα, and wherein said monoclonal antibody or antigen binding fragment, induces death of said human tumor cell.

23. A method of treating ischemia-reperfusion injury, autoimmune disease, autoinflammatory, inflammatory or cardiovascular disease in a human patient comprising administration of a monoclonal antibody or antigen-binding fragment thereof, of any one of claims 1-10.

24. A method of treating cancer in a human patient comprising administration of a monoclonal antibody or antigen-binding fragment thereof, of any one of claims 1-10.

25. The monoclonal antibody or antigen-binding fragment thereof, for use according to any of claims 1-10, for the manufacture of a medicament to prevent, reduce, and/or treat ischemia-reperfusion injury, autoimmune, autoinflammatory, inflammatory or cardiovascular disease in a human patient.

26. The monoclonal antibody or antigen-binding fragment thereof, for use according to any of claims 1-10, for the manufacture of a medicament to treat or reduce a susceptible cancer.

27. A method of assaying CD47 expression in tumor and/or immune cells using a monoclonal antibody, or antigen-binding fragment thereof, of any one of claims 1-10, which specifically binds to an epitope within the sequence of SEQ ID NO:96.

28. The method of claim 27, comprising: obtaining a patient sample, contacting the patient sample with a monoclonal antibody, or antigen-binding fragment thereof, which specifically binds to an epitope within the sequence of SEQ ID NO:96, and assaying for binding of the antibody to the patient sample, wherein binding of the antibody to the patient sample is diagnostic of CD47 expression in a patient sample.

29. The method of claim 27, wherein assaying for binding of the antibody, or antigen binding fragment thereof, to the patient sample utilizes immunohistochemistry labeling of a tissue sample.

30. The method of claim 27, wherein the assaying for binding of the antibody, or antigen binding fragment thereof, to the patient sample utilizes an enzyme linked immunosorbent assay (ELISA).

31. The method of claim 27, wherein the assay for binding of the antibody, or antigen binding fragment thereof, to the patient sample utilizes flow cytometry.

32. The method of claim 27, wherein the patient sample comprises tumor cells, and the assay comprises assaying for the binding of the antibody, or antigen binding fragment thereof, to tumor cells in the patient sample.
